(19) United States
(12) Patent Application Publication
(10) Pub. No.: US 2026/0062761 A1
(43) Pub. Date: Mar. 5, 2026

(54) RNA-BASED METHOD FOR PREDICTING IMMUNE-RELATED ADVERSE EVENTS INDUCED BY CANCER IMMUNOTHERAPY AND EFFICACY OF CANCER IMMUNOTHERAPY

(71) Applicants: THE ASAN FOUNDATION, Seoul (KR); UNIVERSITY OF ULSAN FOUNDATION FOR INDUSTRY COOPERATION, Ulsan (KR); KOREA ADVANCED INSTITUTE OF SCIENCE AND TECHNOLOGY, Daejeon (KR)

(72) Inventors: Sook Ryun PARK, Seoul (KR); Jung Kyoon CHOI, Daejeon (KR); Jinhyeon AN, Daejeon (KR)

(73) Assignees: THE ASAN FOUNDATION, Seoul (KR); UNIVERSITY OF ULSAN FOUNDATION FOR INDUSTRY COOPERATION, Ulsan (KR); KOREA ADVANCED INSTITUTE OF SCIENCE AND TECHNOLOGY, Daejeon (KR)

(21) Appl. No.: 19/384,051

(22) Filed: Nov. 10, 2025

Related U.S. Application Data
(63) Continuation-in-part of application No. PCT/KR2024/006293, filed on May 9, 2024.

(30) Foreign Application Priority Data
May 9, 2023 (KR) 10-2023-0060147

(51) Int. Cl. C12Q 1/6886; G16H 50/30

(52) U.S. Cl. CPC C12Q 1/6886; G16H 50/30; C12Q 2600/106; C12Q 2600/118; C12Q 2600/158

(57) ABSTRACT

The present invention relates to an RNA-based method for predicting the occurrence of immune-related adverse events induced by cancer immunotherapy. As a result of analyzing various factors related to the occurrence of immune-related adverse events induced by cancer immunotherapy in the present invention, it was confirmed that a gene expression-based neutrophil score or immune cell profile is closely associated with the occurrence of immune-related adverse events. Therefore, the neutrophil score or immune cell profile is expected to be effectively used as a biomarker for predicting the occurrence of an immune-related adverse event induced by cancer immunotherapy or efficacy of cancer immunotherapy. In addition, the present invention relates to an RNA-based method for predicting efficacy of cancer immunotherapy. As a result of analyzing various factors related to efficacy to cancer immunotherapy in the present invention, it was confirmed that a gene expression-based tumor necrosis factor (TNF) score or immune cell profile is closely associated with efficacy. Therefore, the TNF score or immune cell profile is expected to be effectively used as a biomarker for predicting efficacy of cancer immunotherapy.

### TECHNICAL FIELD

The present invention relates to an RNA-based method for predicting immune-related adverse events induced by cancer immunotherapy.

Additionally, the present invention relates to an RNA-based method for predicting efficacy of cancer immunotherapy, and more particularly, to a method for predicting efficacy and/or prognosis for cancer immunotherapy by using the expression level of RNA.

This application claims priority to and the benefit of Korean Patent Application No. 10-2023-0060147, filed on May 9, 2023, the disclosure of which is incorporated herein by reference in its entirety.

### BACKGROUND ART

Cancer immunotherapy, representatively immune checkpoint blockade (ICB) treatment, has become one of the major treatment methods for various types of cancer, and its role has expanded from adjuvant therapy to neoadjuvant setting due to immune-related adverse events (irAEs) after cancer immunotherapy. Most early-stage or low-grade irAEs can be managed with corticosteroids or immunosuppressants; however, some irAEs can lead to fatal outcomes or permanent morbidity when not promptly detected or treated. Accordingly, predicting the occurrence of irAEs before ICB treatment (pre-treatment, PRE) or early during treatment (EDT) of cancer immunotherapy is clinically very important, not only for patient management but also from a healthcare cost perspective. In addition, irAEs generally provide an opportunity to understand how autoimmunity develops in response to an immune activator.

Cancer immunotherapy is a therapy that treats cancer by activating a patient's own immune system and is applied as standard therapy and adjuvant treatment across various cancer types after receiving FDA approval. This approach has been gaining attention for its improved therapeutic efficacy and relatively fewer side effects compared to conventional cancer treatments; however, there is heterogeneity in patient responses, with some patients achieving a complete response while others experience a poor treatment prognosis, such as hyperprogressive disease. Therefore, accurate prediction of therapeutic responses at PRE or EDT of ICB treatment would greatly help in developing personalized therapeutic strategies and would benefit patients by minimizing opportunity costs.

Previous irAE studies mainly focused on clinical or biological characteristics measured in peripheral blood. Although a complete blood count (CBC) has been widely studied, conflicting results have been reported in several studies, and this inconsistency indicates that CBC-based biomarkers are easily affected by non-tumor-related factors such as the patient's clinical condition and medical history. Cytokine profiles have also been suggested as predictive markers for irAEs. For example, IL-6 inhibits the differentiation of regulatory T cells and B cells and contributes to the overactivation of the adaptive immune system. Clonal expansion of CD8+ T cells in the peripheral blood has been linked to the development of severe irAEs in patients treated with ipilimumab.

Apart from peripheral blood measurement, tumor mutational burden has been suggested as an indicator of irAE occurrence in an attempt to explain the relatively high irAE occurrence in lung cancer and melanoma. However, it is possible that mutational burden acts as a confounding factor that indirectly increases the risk of irAEs by promoting a therapeutic response to ICB. In addition, TCGA multi-omics data analysis identified LCP1 and ADPGK as predictive biomarkers for irAEs, but the validation of their predictive power was performed on a limited number of lung cancer patients, comparing 14 irAE samples with 14 control samples. These two studies relied on data from the FDA adverse event reporting system (FAERS), but this database was not specifically designed to investigate cancer immunotherapy-related irAEs. For germline variants, polygenic risk scores obtained from genome-wide association studies were applied to atezolizumab-induced skin- or thyroid-related irAEs.

That is, genetic, molecular, and cellular risk factors of irAEs are difficult to identify and require an integrative analysis. The diversity of irAE pathology signifies the multifaceted complexity of the underlying mechanisms and requires a much more comprehensive investigation. However, previous irAE studies were mostly limited to specific drugs (e.g., ipilimumab or atezolizumab), irAE symptoms (e.g., cutaneous autoimmunity), and cancer types (e.g., lung cancer or melanoma), often utilizing a limited number of irAE samples.

Accordingly, in the present invention, by performing a comprehensive analysis of irAEs using integrated multidimensional data including genetic factors, molecular and cell profiles of immune cells, lab data and clinical variables at PRE or EDT of ICB treatment for hundreds of patients with various types of irAEs, it was intended to provide a biomarker for predicting the onset of irAEs induced by cancer immunotherapy such as ICB, and a method of predicting the onset of irAEs using the same.

In addition, in the existing studies, it has been suggested that tumor mutational burden, PD-L1 expression, and the degree of lymphocyte infiltration are associated with the response to cancer immunotherapy. Particularly, a trend was observed in which a higher tumor mutational burden, a higher level of PD-L1 expression, and greater intratumoral lymphocyte infiltration were associated with increased efficacy to cancer immunotherapy. However, many studies were limited to specific cancer types or used only one or two biomarkers.

Therefore, the present invention aimed to discover a biomarker for predicting efficacy of cancer immunotherapy through integrated transcriptome-level analysis of various factors for various types of cancer, hundreds of patient cohort receiving cancer immunotherapy, and to construct a clinical prognosis prediction model based on the biomarker. First, based on transcriptome data derived from peripheral blood before and after treatment, the expression of genes, immune cell profiles, the abundance of immune-related signaling pathways, and the immune repertoire were quantified, and effective biomarkers were quantified through their associations with efficacy, thereby ultimately aiming to develop a precise and broadly applicable model for predicting efficacy to cancer immunotherapy. The biomarker and model for predicting efficacy of cancer immunotherapy developed by this study are expected to contribute to the realization of precision cancer immunotherapy.

### DISCLOSURE

### Technical Problem

The inventors analyzed genetic factors, molecular and cellular profiles of immune cells, laboratory data, and clinical variables related to the occurrence of irAEs before and after ICB treatment, and confirmed the correlation between a gene expression-based neutrophil score and immune cell profile and the occurrence of irAEs, thereby completing the present invention.

Additionally, the inventors analyzed gene expression, immune cell profiles, the abundance of immune-related signaling pathways, and the immune repertoire based on transcriptome cohorts before and after ICB treatment, and confirmed the correlation between a gene expression-based tumor necrosis factor (TNF) score and/or immune cell profile and treatment prognosis, thereby completing the present invention.

Accordingly, an object of the present invention is to provide a method of providing information for predicting the occurrence of an immune-related adverse event (irAE) induced by cancer immunotherapy, comprising measuring the expression of a gene in a biological sample isolated from a subject; and detecting one or more selected from the group consisting of a neutrophil score and immune cell profile.

Another object of the present invention is to provide a composition and kit for predicting the occurrence of an immune-related adverse event (irAE) induced by cancer immunotherapy, comprising an agent for detecting one or more selected from the group consisting of a neutrophil score and immune cell profile.

Another object of the present invention is to provide a method of providing information for predicting efficacy or prognosis of cancer immunotherapy, comprising detecting a tumor necrosis factor (TNF) score and/or immune cell profile in a biological sample isolated from a subject by measuring a gene expression level.

Another object of the present invention is to provide a composition and kit for predicting efficacy or prognosis of cancer immunotherapy, comprising an agent that detects a gene expression level for detecting a tumor necrosis factor (TNF) score and/or immune cell profile.

However, technical problems to be solved in the present invention are not limited to the above-described problems, and other problems which are not described herein will be fully understood by those of ordinary skill in the art from the following descriptions.

### Technical Solution

To achieve the above object, the present invention provides a method of providing information for predicting the occurrence of an immune-related adverse event (irAE) induced by cancer immunotherapy, comprising measuring the expression of a gene in a biological sample isolated from a subject; and detecting one or more selected from the group consisting of a neutrophil score and immune cell profile.

Additionally, the present invention provides a method of providing information for predicting efficacy of cancer immunotherapy, comprising measuring the expression of a gene in a biological sample isolated from a subject; and detecting one or more selected from the group consisting of a neutrophil score and immune cell profile.

In one embodiment of the present invention, the expression of a gene may be measured by one or more methods selected from the group consisting of sequencing, RNA sequencing, and next generation sequencing (NGS), but is not limited thereto.

In another embodiment of the present invention, the immune cell profile may comprise a set of abundance scores for one or more selected from the group consisting of neutrophils, natural killer T cells (NKT), type 1 regulatory T cells (Tr1), type 1 helper T cells (Th1), induced regulatory T cells (iTreg), central memory T cells (Tcm), naive CD4⁺ T cells, effector memory T cells (Tem), and cytotoxic T cells (Tc), but is not limited thereto.

In yet another embodiment of the present invention, the subject may be a cancer patient before or after treatment with cancer immunotherapy, but is not limited thereto.

In yet another embodiment of the present invention, the biological sample may be one or more selected from the group consisting of tissue, cells, whole blood, serum, plasma, saliva, sputum, cerebrospinal fluid, urine, and stool, isolated from the subject, but is not limited thereto.

In yet another embodiment of the present invention, the immune-related adverse event may be one or more selected from the group consisting of a skin adverse event, an endocrine system adverse event, a thyroid gland adverse event, a musculoskeletal system adverse event, a gastrointestinal system adverse event, a neurologic system adverse event, a flu-like symptom, and pneumonia, which occur due to cancer immunotherapy, but is not limited thereto.

In yet another embodiment of the present invention, the method may further comprise predicting that the risk of occurrence of immune-related adverse events is high when one or more selected from the group consisting of a neutrophil score, the abundance score for neutrophils in the immune cell profile, and the abundance score for cytotoxic T cells in the immune cell profile are lower in the biological sample isolated from the subject compared to a control, but is not limited thereto.

In yet another embodiment of the present invention, the method may further comprise predicting that the risk of occurrence of immune-related adverse events is high when the abundance scores for one or more selected from the group consisting of natural killer T cells (NKT), type 1 regulatory T cells (Tr1), type 1 helper T cells (Th1), induced regulatory T cells (iTreg), central memory T cells (Tcm), naive CD4⁺ T cells, and effector memory T cells (Tem) in the immune cell profile are higher in the biological sample isolated from the subject compared to a control, but is not limited thereto.

In yet another embodiment of the present invention, the method may further comprise predicting the risk of occurrence of an immune-related adverse event induced by cancer immunotherapy through a machine learning-based model by detecting one or more selected from the group consisting of a neutrophil score and immune cell profile, but is not limited thereto.

In yet another embodiment of the present invention, the machine learning-based model may be one or more selected from the group consisting of XGBoost and Random forest, but is not limited thereto.

Additionally, the present invention provides a composition for predicting the occurrence of an immune-related adverse event (irAE) induced by cancer immunotherapy, comprising an agent for detecting one or more selected from the group consisting of a neutrophil score and immune cell profile.

In one embodiment of the present invention, the agent for detecting the neutrophil score may comprise a primer or probe that specifically binds to a neutrophil mediated immunity pathway gene set, but is not limited thereto.

In another embodiment of the present invention, the agent for detecting the immune cell profile may comprise a primer or probe that specifically binds to a marker gene set for one or more immune cells selected from the group consisting of neutrophils, natural killer T cells (NKT), type 1 regulatory T cells (Tr1), type 1 helper T cells (Th1), induced regulatory T cells (iTreg), central memory T cells (Tcm), naive CD4⁺ T cells, effector memory T cells (Tem), and cytotoxic T cells (Tc), but is not limited thereto.

Additionally, the present invention provides a kit for predicting the occurrence of an immune-related adverse event (irAE) induced by cancer immunotherapy, comprising the composition.

Additionally, the present invention provides a use of a composition comprising an agent for detecting one or more selected from the group consisting of a neutrophil score and immune cell profile for predicting the occurrence of an immune-related adverse event (irAE) induced by cancer immunotherapy.

Additionally, the present invention provides a use of an agent for detecting one or more selected from the group consisting of a neutrophil score and immune cell profile for preparing an agent for predicting the occurrence of an immune-related adverse event (irAE) induced by cancer immunotherapy.

Additionally, the present invention provides a method of providing information for predicting efficacy or prognosis of cancer immunotherapy, comprising detecting a tumor necrosis factor (TNF) score and/or immune cell profile in a biological sample isolated from a subject by measuring a gene expression level. The method of providing information may further comprise a step of extracting RNA from the biological sample isolated from the subject, but is not limited thereto as long as the step can be comprised in a method for measuring the amount of RNA contained in the biological sample.

Additionally, the present invention provides a composition for predicting efficacy or prognosis of cancer immunotherapy, comprising an agent for detecting a gene expression level for detecting a tumor necrosis factor (TNF) score and/or immune cell profile.

In one embodiment of the present invention, the immune cell profile may comprise a set of abundance scores for one or more selected from the group consisting of CD8⁺ T cells (CD8⁺ T), central memory T cells (Tcm), cytotoxic T cells (Tc), an immune infiltration score, induced regulatory T cells (iTreg), macrophages, neutrophils, type 1 regulatory T cells (Tr1), type 2 helper T cells (Th2), type 17 helper T cells (Th17), and follicular helper T cells (Tfh), but is not limited thereto.

In one embodiment of the present invention, the immune cell profile may be information comprising the expression levels of CD8⁺ T cells (CD8⁺ T), central memory T cells (Tcm), cytotoxic T cells (Tc), an immune infiltration score, induced regulatory T cells (iTreg), macrophages, neutrophils, type 1 regulatory T cells (Tr1), type 2 helper T cells (Th2), type 17 helper T cells (Th17), and follicular helper T cells (Tfh), and the expression levels may be measured as gene expression levels of markers of the immune cells, but are not limited thereto as long as they are methods capable of confirming the expression levels of the immune cells.

In another embodiment of the present invention, the tumor necrosis factor (TNF) score is preferably a value quantified and standardized through Single Sample Gene Set Enrichment Analysis (ssGSEA) using a gene set of the “Negative regulation of tumor necrosis factor mediated signaling pathway (GO: 0010804)” provided by Gene Ontology (http://geneontology.org/), based on gene expression, but is not limited thereto as long as it is a method capable of measuring the expression level of the gene set of the tumor necrosis factor-mediated negative regulation pathway to determine the activation level of tumor necrosis factor.

In yet another embodiment of the present invention, the gene expression level may be measured by a method such as reverse transcriptional polymerase chain reaction (RT-PCR), sequencing, RNA sequencing, microarray, droplet digital polymerase chain reaction (ddPCR), or next generation sequencing (NGS), but is not limited thereto as long as it is a method capable of measuring the gene expression level through RNA.

In yet another embodiment of the present invention, the subject may be a cancer patient before or after treatment with cancer immunotherapy, and the cancer patient after treatment with cancer immunotherapy may be an early-stage patient after treatment, who has received the cancer immunotherapy within 20 weeks, within 15 weeks, preferably within 10 weeks, and more preferably within 8 weeks, but is not limited thereto.

In yet another embodiment of the present invention, the biological sample may be tissue, cells, whole blood, serum, plasma, saliva, sputum, cerebrospinal fluid, urine, or stool isolated from the subject, but is not limited thereto as long as it is a sample containing RNA that can be obtained by a non-invasive method. The sample may be pretreated by a method such as homogenization, filtration, distillation, extraction, concentration, inactivation of interfering components, or addition of reagents before being used for detection or diagnosis.

In yet another embodiment of the present invention, the method may further comprise predicting that efficacy or prognosis for cancer immunotherapy is good when one or more selected from the group consisting of tumor necrosis factor (TNF) score, an immune infiltration score, the abundance score for macrophage, the abundance score for neutrophil, and the abundance score for type 17 helper T cell (Th17) are lower than those of a subject showing no efficacy for cancer immunotherapy; and/or predicting that efficacy or prognosis for cancer immunotherapy is good when the abundance scores for one or more selected from the group consisting of CD8⁺ T cell (CD8⁺ T), central memory T cell (Tcm), cytotoxic T cell (Tc), induced regulatory T cell (iTreg), type 1 regulatory T cell (Tr1), type 2 helper T cell (Th2), and follicular helper T cell (Tfh) are higher than those of a subject showing no efficacy for cancer immunotherapy, wherein the abundance scores for the subject showing no efficacy for cancer immunotherapy may be measured using a biological sample isolated from the subject, and good efficacy or prognosis for cancer immunotherapy may mean that cancer immunotherapy exhibits a therapeutic effect or that the anticancer therapeutic effect is maintained for six months or longer after treatment with cancer immunotherapy, but is not limited thereto as long as it refers to a level generally recognized as exhibiting an anticancer therapeutic effect. The term “therapeutic effect” refers to any action in which cancer and its associated metabolic abnormalities are improved or favorably changed by treatment or administration of cancer immunotherapy.

In yet another embodiment of the present invention, the method may further comprise inputting the measured gene expression level of TNF score and/or the immune cell profile to a machine learning-based model; and predicting efficacy of cancer immunotherapy or a prognosis by automatically classifying the patterns of change compared with the gene expression level of a subject who does not have efficacy for cancer immunotherapy, which is previously input to the machine learning-based model, wherein the step of inputting the gene expression level to the machine learning-based model may be such that the measured gene expression level is automatically input through a computer system.

In yet another embodiment of the present invention, the machine learning-based model may be one or more selected from the group consisting of XGBoost and Random forest, but is not limited thereto, since generally used machine learning-based models may be trained by inputting the gene expression levels of tumor necrosis factor (TNF) score and/or immune cell profile from cancer patients showing efficacy for cancer immunotherapy and cancer patients showing no efficacy thereto, thereby constructing a model for predicting efficacy or prognosis for cancer immunotherapy.

In yet another embodiment of the present invention, the agent for detecting the expression level for determining the tumor necrosis factor (TNF) score may comprise a primer or probe that specifically binds to a gene set of the “Negative regulation of tumor necrosis factor mediated signaling pathway (GO: 0010804),” but is not limited thereto as long as it is an agent capable of measuring the expression level of the gene set of the tumor necrosis factor-mediated negative regulation pathway.

In yet another embodiment of the present invention, the agent for detecting the expression level for determining the immune cell profile may comprise a primer or probe that specifically binds to a marker gene set for one or more immune cells selected from the group consisting of CD8⁺ T cells (CD8⁺ T), central memory T cells (Tcm), cytotoxic T cells (Tc), an immune infiltration score, induced regulatory T cells (iTreg), macrophages, neutrophils, type 1 regulatory T cells (Tr1), type 2 helper T cells (Th2), type 17 helper T cells (Th17), and follicular helper T cells (Tfh), but is not limited thereto as long as it is an agent capable of measuring the expression level of the immune cells.

Additionally, the present invention provides a kit for predicting efficacy or prognosis of cancer immunotherapy, comprising the composition.

Additionally, the present invention provides a use of a composition comprising an agent for detecting one or more selected from the group consisting of a tumor necrosis factor (TNF) score and immune cell profile for predicting efficacy of cancer immunotherapy.

Additionally, the present invention provides a use of an agent for detecting one or more selected from the group consisting of a tumor necrosis factor (TNF) score and immune cell profile for preparing an agent for predicting efficacy of cancer immunotherapy.

### Advantageous Effects

In the present invention, as a result of analyzing various factors associated with the occurrence of an immune-related adverse event (irAE) induced by cancer immunotherapy, it was confirmed that a gene expression-based neutrophil score or immune cell profile is closely related to the occurrence of the immune-related adverse event. Accordingly, the neutrophil score or immune cell profile is expected to be usefully employed as a biomarker for predicting the occurrence of an immune-related adverse event induced by cancer immunotherapy or for predicting efficacy of cancer immunotherapy.

In addition, as a result of analyzing various factors related to efficacy of cancer immunotherapy in the present invention, it was confirmed that a gene expression-based tumor necrosis factor (TNF) score or immune cell profile is closely related to efficacy of cancer immunotherapy. Accordingly, the tumor necrosis factor (TNF) score and/or immune cell profile is expected to be usefully employed as a biomarker for predicting efficacy and/or prognosis for cancer immunotherapy.

### DESCRIPTION OF DRAWINGS

FIG. 1 is a diagram showing the cell type abundance confirmed by PCA analysis using whole blood RNA-seq data according to one embodiment of the present invention.

FIG. 2 is a diagram showing the association between the neutrophil score at pre-treatment (PRE) and early during treatment (EDT) and the occurrence of an immune-related adverse event (irAE) induced by cancer immunotherapy according to one embodiment of the present invention.

FIG. 3 is a diagram comparing the expression levels of neutrophil marker genes between irAE and control groups in PRE and EDT patient samples according to one embodiment of the present invention. *P<0.05, **P<0.01, ***P<0.001.

FIG. 4 is a diagram showing a volcano plot illustrating differentially expressed genes between irAE and control groups in PRE and EDT patient samples according to one embodiment of the present invention.

FIG. 5 is a diagram showing the results of pathway enrichment analysis for genes downregulated in irAE in PRE and EDT patient samples according to one embodiment of the present invention.

FIG. 6 is a diagram showing the degree of pathway enrichment for genes downregulated in irAE across 12 irAE types in PRE and EDT patient samples according to one embodiment of the present invention.

FIG. 7 is a diagram showing the association between the immune cell profile and the occurrence of an immune-related adverse event induced by in PRE and EDT patient samples according to one embodiment of the present invention.

FIG. 8 is a diagram showing the AUC values of training and test sets for predicting the occurrence of an immune-related adverse event induced by cancer immunotherapy using XGBoost and Random forest in PRE and EDT patient samples according to one embodiment of the present invention.

FIG. 9 is a diagram showing the feature importance of XGBoost trained on PRE and EDT patient samples according to one embodiment of the present invention.

FIG. 10 is a diagram showing the results of pathway enrichment analysis for tumor necrosis factor (TNF)-related immune-mediated pathways for genes downregulated in the responder group in PRE and EDT patient samples according to one embodiment of the present invention.

FIG. 11 is a diagram showing the difference in expression levels of neutrophil markers between responder and non-responder groups in PRE and EDT patient samples according to one embodiment of the present invention.

FIG. 12 is a diagram showing the association between the immune cell profile and efficacy of cancer immunotherapy in PRE and EDT patient samples according to one embodiment of the present invention.

FIG. 13 is a diagram showing the AUC values of training and test sets for predicting efficacy of cancer immunotherapy using XGBoost and Random forest in PRE and EDT patient samples according to one embodiment of the present invention.

FIG. 14 is a diagram showing the feature importance of XGBoost trained on PRE and EDT patient samples according to one embodiment of the present invention.

### BEST MODE

In one experimental example of the present invention, among 531 patients treated with ICB, 68 types of irAEs were identified in 276 patients, in which skin-related irAEs were the most frequent type (26%), followed by flu-like (21%) and multiple (all grades) (18%) types (see Experimental Example 1).

In another experimental example of the present invention, RNA sequencing was performed on 531 matched whole blood samples obtained before (PRE) and early during (EDT) ICB treatment to calculate the neutrophil score, and it was confirmed that the neutrophil score was lower in patients who developed irAEs, which was more apparent in early during treatment (EDT) samples. In addition, immune cell types showing a significant association with the occurrence of irAEs were identified, and it was confirmed that this trend was more distinct in the early during treatment (EDT) samples (see Experimental Example 2).

In yet another experimental example of the present invention, an RNA-based model for predicting irAEs was established and validated using XGBoost and Random forest with pre-treatment (PRE) and early during treatment (EDT) samples, and it was confirmed that the test AUC values of the model generated by XGBoost were 0.66 and 0.78 for the pre-treatment and post-treatment samples, respectively, and those of the model generated by Random forest were 0.67 and 0.72 for the pre-treatment and post-treatment samples, respectively. In addition, when the importance of the features of the neutrophil score and immune cell profile was analyzed in the XGBoost model, it was found that cytotoxic T cells were the most important feature before treatment, while neutrophils were the most important feature after treatment (see Experimental Example 3).

In yet another experimental example of the present invention, among 182 patients treated with ICB, 38 patients exhibited efficacy (durable clinical benefits, DCB), whereas 144 patients showed no efficacy (no clinical benefits) (see Experimental Example 4).

In yet another experimental example of the present invention, RNA sequencing was performed on 182 matched whole blood samples obtained before (PRE) and early during (EDT) ICB treatment to calculate the quantitative value of the tumor necrosis factor (TNF)-related signaling pathway, and it was confirmed that the TNF score was lower in responder patients, which was more apparent in the pre-treatment (PRE) samples. In addition, immune cell types showing a significant association with efficacy were identified (see Experimental Example 5).

In yet another experimental example of the present invention, an RNA-based model for predicting therapeutic efficacy was established and validated using XGBoost and Random forest with pre-treatment (PRE) and early during treatment (EDT) samples from ICB-treated patients. As a result, it was confirmed that the test AUC values of the model generated by XGBoost were 0.67 and 0.81 for the pre-treatment and post-treatment samples, respectively, and those of the model generated by Random forest were 0.53 and 0.73 for the pre-treatment and post-treatment samples, respectively. Furthermore, when the importance of the features of the tumor necrosis factor (TNF) score and immune cell profile was analyzed in the XGBoost model, it was found that type 2 helper T cells (Th2) were the most important feature before treatment, while an immune infiltration score was the most important feature after treatment (see Experimental Example 6).

Hereinafter, the present invention will be described in detail.

The present invention provides a method of providing information for predicting the occurrence of an immune-related adverse event (irAE) induced by cancer immunotherapy, comprising measuring the expression of a gene in a biological sample isolated from a subject; and detecting one or more selected from the group consisting of a neutrophil score and immune cell profile.

Additionally, the present invention provides a method for predicting the occurrence of an immune-related adverse event (irAE) induced by cancer immunotherapy, comprising measuring the expression of a gene in a biological sample isolated from a subject; and detecting one or more selected from the group consisting of a neutrophil score and immune cell profile.

Additionally, the present invention provides an analytical method for determining whether a subject receiving cancer immunotherapy has susceptibility or resistance to the occurrence of an immune-related adverse event (irAE) induced by cancer immunotherapy, comprising measuring the expression of a gene in a biological sample isolated from the subject; and detecting one or more selected from the group consisting of a neutrophil score and immune cell profile.

In the present invention, the term “cancer immunotherapy” refers to a treatment using immune anticancer agents. In the present invention, the term “immune anticancer agent” refers to a drug that enhances the body's inherent immune system to increase resistance against cancer. The immune anticancer agent has the advantage of fewer side effects, improved quality of life, and a significantly prolonged survival period for cancer patients, since it treats cancer by enhancing the patient's own immune system. The immune anticancer agent exerts an anticancer effect by enhancing the specificity, memory, and adaptiveness of the immune system. The immune anticancer agent includes, but is not limited to, an agent for immune checkpoint blockade (ICB), an immune cell therapeutic agent, a therapeutic antibody, or an immune enhancer. In the present invention, the agent for immune checkpoint blockade, that is, an immune checkpoint inhibitor, unlike conventional immunotherapeutic agents (such as cytokine therapeutics or cancer vaccines), binds to the binding site between a cancer cell and a T cell to block immune evasion signals, thereby preventing the formation of an immunological synapse and allowing T cells that are not interfered with by immune evasion to destroy cancer cells, and may be one or more selected from the group consisting of nivolumab and pembrolizumab targeting PD-1; atezolizumab, durvalumab, and avelumab targeting PD-L1; and ipilimumab and tremelimumab targeting CTLA-4, but is not limited thereto.

In the present invention, the term “immune-related adverse event (irAE)” refers to various adverse events that occur during treatment with cancer immunotherapy, including inflammatory responses associated with the activation of the autoimmune system.

In the present invention, the immune-related adverse event may be one or more selected from the group consisting of a skin adverse event, an endocrine system adverse event, a thyroid gland adverse event, a musculoskeletal system adverse event, a gastrointestinal system adverse event, a neurologic system adverse event, a flu-like symptom, and pneumonia caused by cancer immunotherapy, but is not limited thereto. According to one example or experimental example of the present invention, the immune-related adverse event may occur in combination with two or more symptoms, and the severity thereof may be classified into three grades. For example, in the present invention, depending on the severity of the immune-related adverse event, cases with three or more types of immune-related adverse events are denoted as “Multiple G>=1,” cases with three or more types of grade 2 or higher are denoted as “Multiple G>=2,” cases with any type of grade 3 or higher or critical types of grade 2 or higher are denoted as “Critical,” and all cases falling within the immune-related adverse event category may be denoted as “Any.”

In the present invention, in the method of providing information for predicting the occurrence of an immune-related adverse event induced by cancer immunotherapy or the method for predicting the occurrence of an immune-related adverse event induced by cancer immunotherapy, the term “detection” refers to both measuring and confirming the presence (expression) of a target substance and measuring and confirming a change in the presence level (expression level) of the target substance. In the same context, in the present invention, detecting the neutrophil score or immune cell profile means measuring whether neutrophil-mediated immune pathway genes, neutrophils, or immune cells are present (that is, measuring their presence or absence), or measuring qualitative or quantitative changes in neutrophil-mediated immune pathway genes, neutrophils, or immune cells. The measurement may be performed without limitation, including both qualitative and quantitative methods (analyses). The types of qualitative and quantitative methods for measuring the presence of neutrophil-mediated immune pathway genes, neutrophils, or immune cells are well known to those skilled in the art, and the experimental methods described in the present specification are included therein.

In the present invention, in the method of providing information for predicting the occurrence of an immune-related adverse event induced by cancer immunotherapy or the method for predicting the occurrence of an immune-related adverse event induced by cancer immunotherapy, the term “subject” may include a cancer patient to be treated with cancer immunotherapy, that is, both a cancer patient before treatment with cancer immunotherapy and a cancer patient after treatment with cancer immunotherapy, and the cancer patient after treatment with cancer immunotherapy may be an early-stage patient after treatment, but is not limited thereto.

In the present invention, in the method of providing information for predicting the occurrence of an immune-related adverse event induced by cancer immunotherapy or the method for predicting the occurrence of an immune-related adverse event induced by cancer immunotherapy, the term “control group” may refer to a normal subject or a cancer patient who has not exhibited immune-related adverse events after treatment with cancer immunotherapy, and according to one embodiment of the present invention, the control group may be a cancer patient who has not exhibited immune-related adverse events after treatment with cancer immunotherapy, but is not limited thereto.

As used herein, the term “cancer” refers to a disease characterized by uncontrolled cell growth, in which abnormal cell proliferation leads to the formation of a mass of cells called a tumor that invades surrounding tissues and, in severe cases, metastasizes to other organs of the body. Academically, it is also referred to as a neoplasm. Even when treated by surgery, radiation, or chemotherapy, cancer often cannot be fundamentally cured, causes suffering to patients, and ultimately leads to death, being an intractable chronic disease. The causes of cancer are diverse and can be classified into internal and external factors. Although the exact mechanisms by which normal cells are transformed into cancer cells have not been fully elucidated, it is known that many cancers are caused or influenced by external factors such as environmental conditions. Internal factors include genetic and immunological factors, while external factors include chemical substances, radiation, and viruses. Genes involved in cancer development include oncogenes and tumor suppressor genes, and cancer occurs when the balance between these genes is disrupted by the aforementioned internal or external factors. In the present invention, the term “cancer” encompasses primary cancer, cancer treated with radiotherapy, cancer not treated with radiotherapy, metastatic cancer, and recurrent cancer. In addition, the composition of the present invention may be particularly suitable for the treatment of non-immunogenic tumors or cancers.

In the present invention, cancer may include all types of solid cancers and all types of hematologic cancers. By way of non-limiting example, the cancer of the present invention may be one or more selected from the group consisting of adenocarcinoma, choroidal melanoma, acute leukemia, acoustic neurinoma, ampullary carcinoma, anal carcinoma, astrocytoma, basal cell carcinoma, pancreatic cancer, desmoid tumor, bladder cancer, bronchial carcinoma, non-small cell lung cancer (NSCLC), small cell lung cancer (SCLC), breast cancer, Burkitt's lymphoma, corpus cancer, carcinoma of unknown primary (CUP) syndrome, colorectal cancer, small intestine cancer, small intestinal tumors, ovarian cancer, endometrial carcinoma, ependymoma, epithelial cancer types, Ewing's tumors, gastrointestinal tumors, gastric cancer, biliary cancer, gallbladder cancer, gall bladder carcinomas, uterine cancer, cervical cancer, cervix, glioblastomas, gynecologic tumors, ear, nose and throat tumors, hematologic neoplasias, hairy cell leukemia, urethral cancer, skin cancer, skin testis cancer, brain tumors, gliomas, brain metastases, testicle cancer, hypophysis tumor, carcinoids, Kaposi's sarcoma, laryngeal cancer, germ cell tumor, bone cancer, colorectal carcinoma, head and neck tumors (tumors in the otorhinolaryngology region), colon carcinoma, craniopharyngiomas, oral cancer (cancers of the oral region and lips), cancers of the central nervous system, liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumors of the gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, squamous cell carcinoma of the head and neck (SCCHN), prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumors, urethral cancer, urologic tumors, urothelial carcinoma, vulva cancer, wart appearance, soft tissue tumors, soft tissue sarcoma, Wilm's tumor, cervical carcinoma, and tongue cancer, but is not limited thereto.

In the present invention, the cancer may be one or more selected from the group consisting of lung cancer including non-small cell lung cancer and small cell lung cancer, esophageal cancer, hepatocellular carcinoma, gastric cancer, breast cancer, bladder cancer, kidney cancer, bile duct cancer, urethral cancer, head and neck cancer, melanoma, colorectal cancer, gallbladder cancer, pancreatic cancer, ampullary cancer, neuroendocrine carcinoma, paraganglioma, ovarian cancer, uterine cancer, prostate cancer, thymic cancer, and cerebral angiosarcoma, but is not limited thereto.

In the present invention, the biological sample may be one or more selected from the group consisting of tissue, cells, whole blood, serum, plasma, saliva, sputum, cerebrospinal fluid, urine, and feces isolated from a subject, and according to one embodiment or experimental example of the present invention, it may be whole blood, but is not limited thereto.

In the present invention, in the method of providing information for predicting the occurrence of an immune-related adverse event induced by cancer immunotherapy or the method for predicting the occurrence of an immune-related adverse event induced by cancer immunotherapy, the term “measurement of expression or expression level” refers to a process of determining the presence and/or expression amount of mRNA coded from neutrophil-mediated immune pathway genes, or the presence and/or expression amount of mRNA coded from marker genes associated with immune cells, in a biological sample to confirm information related to a neutrophil score or immune cell profile.

In the present invention, the step of measuring gene expression may be performed using conventional methods known in the art, for example, by one or more methods selected from the group consisting of reverse transcriptional polymerase chain reaction (RT-PCR), sequencing, RNA sequencing, microarray, droplet digital polymerase chain reaction (ddPCR), and next generation sequencing (NGS), but is not limited thereto.

In the present invention, the gene expression refers to a quantified value obtained by comparing reads acquired from RNA sequencing with a human reference genome, and according to one embodiment of the present invention, the human reference genome may be GRCh37 (hg19), but is not limited thereto.

In the present invention, the neutrophil score may be measured using a method known in the art. For example, the neutrophil score may refer to a value quantified and normalized through single sample gene set enrichment analysis (ssGSEA) based on gene expression using a neutrophil mediated immunity pathway (GO: 0002446) gene set provided by Gene Ontology (http://geneontology.org/), but is not limited thereto. At this time, the quantified value of the neutrophil mediated immunity pathway gene set may represent the total gene expression amount obtained by evaluating the integrated expression of the gene set, and the quantification and normalization may be calculated using the ssGSEA method (Barbie et al., Nature, 2009, PMID: 19847166; genepattern.org/modules/docs/ssGSEAProjection/5 #gsc.tab-0).

In the present invention, in the method of providing information for predicting the occurrence of an immune-related adverse event induced by cancer immunotherapy or the method for predicting the occurrence of an immune-related adverse event induced by cancer immunotherapy, the immune cell profile may comprise a set of abundance scores for one or more selected from the group consisting of neutrophils, natural killer T cells (NKT), type 1 regulatory T cells (Tr1), type 1 helper T cells (Th1), induced regulatory T cells (iTreg), central memory T cells (Tcm), naive CD4⁺ T cells, effector memory T cells (Tem), and cytotoxic T cells (Tc), but is not limited thereto. In the present invention, the immune cell profile may be measured using a method known in the art, for example, may be measured using ImmuCellAI (Miao, Y. et al., Adv. Sci. 7, 1902880; http://bioinfo.life.hust.edu.cn/ImmuCellAI), but is not limited thereto as long as the method can measure an immune cell profile.

In the present invention, in the method of providing information for predicting the occurrence of an immune-related adverse event induced by cancer immunotherapy or the method for predicting the occurrence of an immune-related adverse event induced by cancer immunotherapy, the abundance score may be a quantitative value of the total gene expression amount of marker gene sets for each immune cell provided by ImmuCellAI. The immune cell profile may be measured by quantifying the total gene expression amount of immune cell marker gene sets provided by ImmuCellAI, and the marker gene sets for each immune cell are as follows:

Marker genes for neutrophils: IL18RAP, BMX, BTNL8, CASP5, CLC, CXCR1, CXCR2, FCGR3B, FPR2, HSPA6, MEFV, PADI4, S100A12, TREML2, TRPM6, SIGLEC5, CREB5, ALPL, AATK, CEACAM3, CSF2RB, CSF3R, FBXO38, MAK, PAK2, TOP1, UBXN2B, and VNN3.

Marker genes for natural killer T cells (NKT): CASP5, DOLK, GMIP, PRR5L, SGCA, SLAMF1, TCOF1, and TGFBR2.

Marker genes for type 1 regulatory T cells (Tr1): TNFRSF4, CD4, CCR4, CD28, and LAX1.

Marker genes for type 1 helper T cells (Th1): IFNG, IL2, MNAT1, SLAMF1, STAT1, EIF2B2, APBB2, CCL4, CTLA4, GGT1, LTA, SYNGR3, and TACO1.

Marker genes for induced regulatory T cells (iTreg): FOXP3, STAT5A, CCR8, CD5, HS3ST3B1, TTN, CTLA4, FASLG, ICOS, CCR3, GALNT8, NFATC3, SIT1, CD28, IL10RA, PPM1B, ATG2B, CCR4, and ZFYVE9.

Marker genes for central memory T cells (Tcm): CORO7, ATM, GMEB2, SNRPN, ADSL, ITK, TFAP4, NAA16, LY9, CYLD, GIMAP4, PURA, DVL1, RPP38, LRIG2, IL7R, CDKN2A1P, APBB1, IPCEF1, CD247, CD40LG, TRADD, CD3E, TPR, ARID5B, UBASH3A, NCK1, SPTAN1, GPR171, and CD5.

Marker genes for naive CD4⁺ T cells: CD2, CD3G, CD4, GIMAP6, GLG1, HMOX2, IL7R, ITK, LIMD2, LY9, NAA16, OBSCN, PACS1, PLCL1, RPL14, SNPH, TPP2, TRAF1, ZBTB40, CD40LG, and SEPT9.

Marker genes for effector memory T cells (Tem): APBA3, CD160, CD2, CD8B, CDK10, CDKN2A1P, CHST12, COG4, CX3CR1, DHX16, EWSR1, GIMAP6, GPR171, GZMK, HOMX2, IKZF3, ITK, KLRD1, KLRG1, MAPKAPK5, MORC2, MRFAP1L1, PLCG1, PSMC5, RNF167, SBF1, SF3B2, SLAMF1, TBCD, USP47, ZFYVE9, and ZNF549.

Marker genes for cytotoxic T cells (Tc): KNG1, PSORS1C2, BLNK, SCN3A, TNFRSF10C, ITGAM, KLRK1, CD8A, GNLY, GZMH, PTGDR2, CD8B, GZMA, and PRF1.

In the present invention, in the method of providing information for predicting the occurrence of an immune-related adverse event induced by cancer immunotherapy or the method for predicting the occurrence of an immune-related adverse event induced by cancer immunotherapy, the method may further comprise predicting that the risk of developing an immune-related adverse event is high when one or more selected from the group consisting of a neutrophil score, the abundance score for neutrophils in the immune cell profile, and the abundance score for cytotoxic T cells in the immune cell profile are lower than those of the control group, but is not limited thereto.

In the present invention, in the method of providing information for predicting the occurrence of an immune-related adverse event induced by cancer immunotherapy or the method for predicting the occurrence of an immune-related adverse event induced by cancer immunotherapy, the method may further comprise predicting that the risk of developing an immune-related adverse event is high when the abundance scores for one or more selected from the group consisting of natural killer T cells (NKT), type 1 regulatory T cells (Tr1), type 1 helper T cells (Th1), induced regulatory T cells (iTreg), central memory T cells (Tcm), naive CD4⁺ T cells, and effector memory T cells (Tem) in the immune cell profile are higher than those of the control group, but is not limited thereto.

In the present invention, in the method of providing information for predicting the occurrence of an immune-related adverse event induced by cancer immunotherapy or the method for predicting the occurrence of an immune-related adverse event induced by cancer immunotherapy, the method may further comprise predicting the risk of occurrence of an immune-related adverse event induced by cancer immunotherapy through a machine learning-based model by detecting one or more selected from the group consisting of a neutrophil score and immune cell profile, but is not limited thereto.

In the present invention, in the method of providing information for predicting the occurrence of an immune-related adverse event induced by cancer immunotherapy or the method for predicting the occurrence of an immune-related adverse event induced by cancer immunotherapy, the machine learning-based model may be one or more selected from the group consisting of XGBoost and Random forest, but is not limited thereto.

In the present invention, “XGBoost,” an abbreviation of Extreme Gradient Boosting, is a type of ensemble boosting technique. It constructs a model by sequentially compensating for the errors of previous models, using the difference (loss) between actual and predicted values from the previous model as training data and applying gradient-based optimization to correct the errors.

In the present invention, “Random forest” is a type of ensemble learning method used for classification and regression analysis, which operates by outputting the class (for classification) or the mean prediction (for regression) from multiple decision trees constructed during the training process.

Additionally, the present invention provides a method of providing information for predicting efficacy of cancer immunotherapy, comprising measuring the expression of a gene in a biological sample isolated from a subject; and detecting one or more selected from the group consisting of a neutrophil score and immune cell profile.

Additionally, the present invention provides a method for predicting efficacy of cancer immunotherapy, comprising measuring the expression of a gene in a biological sample isolated from a subject; and detecting one or more selected from the group consisting of a neutrophil score and immune cell profile.

An analytical method for determining whether a subject receiving cancer immunotherapy has susceptibility or resistance to cancer immunotherapy, comprising measuring the expression of a gene in a biological sample isolated from the subject; and detecting one or more selected from the group consisting of a neutrophil score and immune cell profile.

In the present invention, when one or more selected from the group consisting of a neutrophil score, the amount of neutrophils in the immune cell profile, and the amount of cytotoxic T cells in the immune cell profile in a biological sample isolated from a subject are lower than those of the control group, it may be predicted that efficacy of cancer immunotherapy is low, but is not limited thereto.

In the present invention, when the abundance scores for one or more selected from the group consisting of natural killer T cells (NKT), type 1 regulatory T cells (Tr1), type 1 helper T cells (Th1), induced regulatory T cells (iTreg), central memory T cells (Tcm), naive CD4⁺ T cells, and effector memory T cells (Tem) in the immune cell profile in a biological sample isolated from a subject are higher than those of the control group, it may be predicted that efficacy of cancer immunotherapy is high, but is not limited thereto.

Additionally, the present invention provides a composition for predicting the occurrence of an immune-related adverse event (irAE) induced by cancer immunotherapy, comprising an agent for detecting one or more selected from the group consisting of a neutrophil score and immune cell profile.

In the present invention, in the composition for predicting the occurrence of an immune-related adverse event induced by cancer immunotherapy, the agent is not limited in type as long as it is capable of detecting a neutrophil score or an immune cell profile, and all agents used in methods known in the art for detecting a neutrophil score or an immune cell profile may be included. For example, the agent may include components, compositions, solutions, or devices conventionally required for single sample gene set enrichment analysis (ssGSEA) based on gene expression using a neutrophil mediated immunity pathway gene set provided by Gene Ontology (http://geneontology.org/) as a method for detecting the neutrophil score, and components, compositions, solutions, or devices conventionally required for ImmuCellAI (Miao, Y. et al., Adv. Sci. 7, 1902880) as a method for detecting the immune cell profile. According to one embodiment of the present invention, the agent for detecting the neutrophil score may comprise a primer or probe that specifically binds to a neutrophil mediated immunity pathway gene set, and the agent for detecting the immune cell profile may comprise a primer or probe that specifically binds to one or more marker genes selected from the group consisting of neutrophils, natural killer T cells (NKT), type 1 regulatory T cells (Tr1), type 1 helper T cells (Th1), induced regulatory T cells (iTreg), central memory T cells (Tcm), naive CD4⁺ T cells, effector memory T cells (Tem), and cytotoxic T cells (Tc), but is not limited thereto.

In the present invention, the term “primer” refers to a short single-stranded oligonucleotide that serves as a starting point for DNA synthesis. The primer specifically binds to a template polynucleotide under appropriate buffer and temperature conditions, and DNA synthesis occurs as a DNA polymerase extends the primer by adding nucleoside triphosphates complementary to the template DNA. Primers are generally composed of 15 to 30 nucleotides, and the melting temperature (Tm) at which they bind to the template strand varies depending on their nucleotide composition and length. The sequence of the primer does not necessarily need to be completely complementary to a portion of the template sequence, as long as it has a length and complementarity suitable for the purpose of amplifying a specific region of mRNA or cDNA through DNA synthesis to measure the amount of mRNA. Accordingly, in the present invention, a primer pair may be easily designed by referring to the nucleotide sequence of the gene or the cDNA or genomic DNA of its mRNA. The primer for the amplification reaction consists of a pair that complementarily binds to both ends of the specific region of the mRNA to be amplified, one on the sense strand (template) and the other on the antisense strand.

In the present invention, the term “probe” refers to a fragment of a polynucleotide, such as RNA or DNA, ranging from several to several hundred base pairs in length, which can specifically bind to mRNA, cDNA (complementary DNA), or DNA of a particular gene, and is labeled so that the presence, absence, or expression level of the target mRNA or cDNA to which it binds can be detected. The selection of the probe and hybridization conditions may be appropriately determined according to techniques well known in the art. The probe may be used in diagnostic methods for detecting alleles (or allelomorphs). Such diagnostic methods include detection methods based on nucleic acid hybridization, such as Southern blotting, and may also be provided in a form pre-bound to the substrate of a DNA chip in methods utilizing DNA chips.

In the present invention, the primer or probe may be chemically synthesized using a phosphoramidite solid-support synthesis method or other well-known techniques. In addition, the primer or probe may be variously modified according to methods known in the art, as long as such modifications do not interfere with hybridization to the target polynucleotide to be detected. Examples of such modifications include methylation, capping, substitution with one or more homologs of natural nucleotides, and modification of inter-nucleotide linkages, such as uncharged linkages (e.g., methyl phosphonate, phosphotriester, phosphoramidate, carbamate) or charged linkages (e.g., phosphorothioate, phosphorodithioate), as well as conjugation with labeling materials such as fluorescent or enzymatic tags.

In the present invention, the primer or probe is not limited to a specific sequence as long as it is capable of detecting a neutrophil score or an immune cell profile.

Additionally, the present invention provides a kit for predicting the occurrence of an immune-related adverse event (irAE) induced by cancer immunotherapy or a kit for predicting efficacy of cancer immunotherapy, comprising the composition.

In the kit for predicting the occurrence of an immune-related adverse event (irAE) induced by cancer immunotherapy or the kit for predicting efficacy of cancer immunotherapy according to the present invention, the term “kit” refers to a tool that allows prediction of the occurrence of an immune-related adverse event induced by cancer immunotherapy or prediction of efficacy of cancer immunotherapy in a cancer patient by including an agent for detecting a neutrophil score or an immune cell profile. The kit of the present invention may further include, in addition to the agent, other components, compositions, solutions, or devices conventionally required for the measurement or detection methods thereof.

In the kit for predicting the occurrence of an immune-related adverse event (irAE) induced by cancer immunotherapy or the kit for predicting efficacy of cancer immunotherapy according to the present invention, the kit may comprise a container, an instruction manual, and an agent for detecting a neutrophil score or an immune cell profile. The container may serve to package the agent and may also serve to store and secure it. The material of the container may take the form of, for example, a bottle, tub, sachet, envelope, tube, or ampoule, and may be formed partially or entirely from plastic, glass, paper, foil, wax, or the like. The container may be equipped with a cap that is initially a part of the container or is completely or partially detachable and attachable to the container by mechanical, adhesive, or other means, and may also be equipped with a stopper that allows access to the contents with a syringe needle. The kit may include an external package, and the external package may include instructions for use of the components.

Additionally, the present invention provides a use of a composition comprising an agent for detecting one or more selected from the group consisting of a neutrophil score and immune cell profile for predicting the occurrence of an immune-related adverse event (irAE) induced by cancer immunotherapy or for predicting efficacy of cancer immunotherapy.

Additionally, the present invention provides a use of an agent for detecting one or more selected from the group consisting of a neutrophil score and immune cell profile for preparing an agent for predicting the occurrence of an immune-related adverse event (irAE) induced by cancer immunotherapy or for preparing an agent for predicting efficacy of cancer immunotherapy.

Additionally, the present invention provides a method of providing information for determining or analyzing whether a subject is highly susceptible to prediction of the occurrence of an immune-related adverse event induced by cancer immunotherapy or to prediction of efficacy of cancer immunotherapy, wherein after measuring gene expression from RNA obtained from biological samples collected before and after treatment from a subject suspected of having a high risk of developing an immune-related adverse event after cancer immunotherapy or a subject suspected of having low efficacy for cancer immunotherapy, and from a control group, when one or more low values selected from the group consisting of a neutrophil score, the abundance score for neutrophils in the immune cell profile, and the abundance score for cytotoxic T cells in the immune cell profile are measured, or when high values of the abundance scores for one or more selected from the group consisting of natural killer T cells (NKT), type 1 regulatory T cells (Tr1), type 1 helper T cells (Th1), induced regulatory T cells (iTreg), central memory T cells (Tcm), naive CD4⁺ T cells, and effector memory T cells (Tem) in the immune cell profile are measured, it is considered that the subject has a high risk of developing immune-related adverse events after cancer immunotherapy or low efficacy for cancer immunotherapy.

Additionally, the present invention provides a method for treating cancer, comprising:
measuring gene expression in a biological sample isolated from a subject;
detecting one or more selected from the group consisting of a neutrophil score and immune cell profile;
predicting that the risk of developing an immune-related adverse event is low when one or more selected from the group consisting of a neutrophil score, the abundance score for neutrophils in the immune cell profile, and the abundance score for cytotoxic T cells in the immune cell profile are higher than those of a subject who developed an immune-related adverse event induced by cancer immunotherapy in the biological sample isolated from the subject, or when the abundance scores for one or more selected from the group consisting of natural killer T cells (NKT), type 1 regulatory T cells (Tr1), type 1 helper T cells (Th1), induced regulatory T cells (iTreg), central memory T cells (Tcm), naive CD4⁺ T cells, and effector memory T cells (Tem) in the immune cell profile are lower than those of a subject who developed an immune-related adverse event induced by cancer immunotherapy in the biological sample isolated from the subject; and
treating the subject predicted to have a low risk of developing the immune-related adverse event with cancer immunotherapy.

In the present invention, the cancer immunotherapy may comprise administering one or more selected from the group consisting of an agent for immune checkpoint blockade (ICB), an immune cell therapeutic agent, a therapeutic antibody, and an immune enhancer.

Additionally, the present invention provides a method of providing information for predicting efficacy or prognosis of cancer immunotherapy, comprising measuring gene expression levels in a biological sample isolated from a subject to detect a tumor necrosis factor (TNF) score and/or immune cell profile.

Additionally, the present invention provides a method for predicting efficacy or prognosis of cancer immunotherapy, comprising measuring gene expression levels in a biological sample isolated from a subject to detect a tumor necrosis factor (TNF) score and/or immune cell profile.

Additionally, the present invention provides an analytical method for determining whether a subject receiving cancer immunotherapy has susceptibility or resistance to cancer immunotherapy, comprising:
detecting a tumor necrosis factor (TNF) score and/or immune cell profile in a biological sample isolated from the subject by measuring a gene expression level,
wherein the immune cell profile comprises one or more selected from the group consisting of CD8⁺ T cells, central memory T cells (Tcm), cytotoxic T cells (Tc), an immune infiltration score, an induced regulatory T cells (iTreg), macrophages, neutrophils, type 1 regulatory T cells (Tr1), type 2 helper T cells (Th2), type 17 helper T cells (Th17), and follicular helper T cells (Tfh).

In the present invention, the clinical response (DCB) group refers to a patient group showing a complete response (CR), a partial response (PR), or a stable disease (SD) exhibiting a progression free survival (PFS) of 6 months or more or an overall survival (OS) of 1 year or more, according to RECIST 1.1 criteria. Other groups are classified as non-clinical benefit (NCB) groups.

In the present invention, in the method of providing information for predicting efficacy or prognosis of cancer immunotherapy or the method for predicting efficacy or prognosis of cancer immunotherapy, the term “detecting” refers to both measuring and confirming the presence (expression) of a target substance, or measuring and confirming changes in the presence level (expression level) of the target substance. In the same context, in the present invention, detecting the tumor necrosis factor (TNF) score or immune cell profile means measuring the amount of tumor necrosis factor or determining the presence of immune cells (i.e., measuring the presence or absence thereof), or measuring qualitative or quantitative changes in the levels of the tumor necrosis factor or immune cells. The measurement may be performed without limitation and includes both qualitative and quantitative methods (analyses). The types of qualitative and quantitative methods for determining the presence of tumor necrosis factor or immune cells are well known in the art, and the experimental methods described in the present specification are included therein. The tumor necrosis factor (TNF) score refers to the expression value for the gene set of the negative regulation of tumor necrosis factor mediated signaling pathway (GO: 0010804), and the gene set may include genes related to nuclear-transcribed mRNA catabolic process, dopamine neurotransmitter receptor activity, coupled via Gi, GINS complex, astral microtubule nucleation, Las1 complex, PAM complex, Tim23 associated import motor, viral translational termination-reinitiation, positive regulation of microtubule nucleation, activation of microtubule nucleation, regulation of microtubule nucleation, digestive tract mesoderm development, gonadal mesoderm development, G protein-coupled opsin signaling pathway, CoA-synthesizing protein complex, photosystem I, Tapasin-ERp57 complex, optic cup morphogenesis involved in camera-type eye development, Dom34-Hbs1 complex, bBAF complex, ATAC complex, Ragulator complex, and SAGA complex, but is not limited thereto as long as it is included in the gene set of GO: 0010804.

In the present invention, in the method of providing information for predicting efficacy or prognosis of cancer immunotherapy or the method for predicting efficacy or prognosis of cancer immunotherapy, the term “measuring expression level” refers to the process of determining the presence and/or expression amount of mRNA encoded from genes associated with tumor necrosis factor, or the presence and/or expression amount of mRNA encoded from marker genes associated with immune cells, in a biological sample, in order to obtain information regarding the tumor necrosis factor (TNF) score or immune cell profile.

In the present invention, the tumor necrosis factor (TNF) score may be measured using methods known in the art, and, for example, the TNF score may refer to a quantified and normalized value obtained through single sample gene set enrichment analysis (ssGSEA) based on gene expression using the gene set of the negative regulation of tumor necrosis factor mediated signaling pathway (GO: 0010804) provided by Gene Ontology (http://geneontology.org/), but is not limited thereto. At this time, the quantified value of the negative regulation of tumor necrosis factor mediated signaling pathway gene set may represent the total gene expression amount obtained by assessing the integrated expression of the gene set, and the quantification and normalization may be calculated using the ssGSEA method (Barbie et al., Nature, 2009, PMID: 19847166; genepattern.org/modules/docs/ssGSEAProjection/5 #gsc.tab=0).

In the present invention, in the method of providing information for predicting efficacy or prognosis of cancer immunotherapy or the method for predicting efficacy or prognosis of cancer immunotherapy, the immune cell profile may comprise a set of abundance scores one or more immune cells selected from the group consisting of CD8⁺ T cells (CD8⁺ T), central memory T cells (Tcm), cytotoxic T cells (Tc), an immune infiltration score, induced regulatory T cells (iTreg), macrophages, neutrophils, type 1 regulatory T cells (Tr1), type 2 helper T cells (Th2), type 17 helper T cells (Th17), and follicular helper T cells (Tfh), but is not limited thereto. In the present invention, the immune cell profile may be measured using methods known in the art, for example, measured by ImmuCellAI (Miao, Y. et al., Adv. Sci. 7, 1902880; http://bioinfo.life.hust.edu.cn/ImmuCellAI), but is not limited thereto as long as it is a method capable of measuring an immune cell profile.

In the present invention, in the method of providing information for predicting efficacy or prognosis of cancer immunotherapy or the method for predicting efficacy or prognosis of cancer immunotherapy, the abundance score may be a quantitative value of the total gene expression amount of marker gene sets for each immune cell provided by ImmuCellAI. The immune cell profile may be measured by quantifying the total gene expression of immune cell marker gene sets provided by ImmuCellAI (Adv. Sci. 2020, 7, 1902880), and the marker gene sets for each immune cell are as follows:

Marker genes for CD8⁺ T cells (CD8⁺ T): CD27, CD8B, CLUAP1, CRTAM, FKTN, KLRG1, LY9, PLCG1, RING1, SF1, SIRPG, TSPAN32, ARHGEF1, EEF1D, PPPIR2, CTSW, FBXW4, ZNF611, GZMH, CD8A, TTN, CD7 and CX3CR1.

Marker genes for central memory T cells (Tcm): CORO7, ATM, GMEB2, SNRPN, ADSL, ITK, TFAP4, NAA16, LY9, CYLD, GIMAP4, PURA, DVL1, RPP38, LRIG2, IL7R, CDKN2A1P, APBB1, IPCEF1, CD247, CD40LG, TRADD, CD3E, TPR, ARID5B, UBASH3A, NCK1, SPTAN1, GPR171 and CD5.

Marker genes for cytotoxic T cells (Tc): KNG1, PSORS1C2, BLNK, SCN3A, TNFRSF10C, ITGAM, KLRK1, CD8A, GNLY, GZMH, PTGDR2, CD8B, GZMA and PRF1.

Marker genes for induced regulatory T cells (iTreg): FOXP3, STAT5A, CCR8, CD5, HS3ST3B1, TTN, CTLA4, FASLG, ICOS, CCR3, GALNT8, NFATC3, SIT1, CD28, IL10RA, PPM1B, ATG2B, CCR4 and ZFYVE9.

Marker genes for macrophages: ARPC4, ATP6V0E1, BPI, C1QA, C1QB, CAMP, CHIT1, CLEC5A, CLIP1, CSF1R, CYBB, FGR, GGA1, GRB2, IFNAR1, IGSF6, IL17RA, LILRA2, MARCO, MMP8, MS4A6A, OTUD4, PSME1 and RENBP.

Marker genes for neutrophils: IL18RAP, BMX, BTNL8, CASP5, CLC, CXCR1, CXCR2, FCGR3B, FPR2, HSPA6, MEFV, PADI4, S100A12, TREML2, TRPM6, SIGLEC5, CREB5, ALPL, AATK, CEACAM3, CSF2RB, CSF3R, FBXO38, MAK, PAK2, TOP1, UBXN2B, and VNN3.

Marker genes for type 1 regulatory T cells (Tr1): TNFRSF4, CD4, CCR4, CD28, and LAX1.

Marker genes for type 2 helper T cells (Th2): GZMK, IL4, GATA3, GSTA4 and SLC25A44.

Marker genes for type 17 helper T cells (Th17): IL1R1, RORC, CD4, IL21 and IL17RA.

Marker genes for follicular helper T cells (Tfh): CA8, CD2, CD3G, GZMM, ITK, KLRB1 LTA, MAP4K1, ST8SIA1, TRAC, TRAV9-2, TRIB2 and UBASH3A.

In the present invention, in the method of providing information for predicting efficacy or prognosis of cancer immunotherapy or the method for predicting efficacy or prognosis of cancer immunotherapy, the method may further comprise predicting that efficacy or prognosis of cancer immunotherapy is good when one or more selected from the group consisting of a tumor necrosis factor (TNF) score, an immune infiltration score, the abundance score for macrophages, the abundance score for neutrophils, and the abundance score for type 17 helper T cells (Th17) in the immune cell profile in a biological sample isolated from a subject are lower than the gene expression levels of a subject having no efficacy for cancer immunotherapy, but is not limited thereto.

In the present invention, in the method of providing information for predicting efficacy or prognosis of cancer immunotherapy or the method for predicting efficacy or prognosis of cancer immunotherapy, the method may further comprise predicting that efficacy or prognosis of cancer immunotherapy is good when the abundance scores for one or more selected from the group consisting of CD8⁺ T cells (CD8⁺ T), central memory T cells (Tcm), cytotoxic T cells (Tc), induced regulatory T cells (iTreg), type 1 regulatory T cells (Tr1), type 2 helper T cells (Th2), and follicular helper T cells (Tfh) in the immune cell profile in a biological sample isolated from a subject are higher than those of a subject having no efficacy for cancer immunotherapy, but is not limited thereto.

In the present invention, in the method of providing information for predicting efficacy or prognosis of cancer immunotherapy or the method for predicting efficacy or prognosis of cancer immunotherapy, the method may further comprise predicting the degree of efficacy of cancer immunotherapy through a machine learning-based model by detecting a tumor necrosis factor (TNF) score or immune cell profile, but is not limited thereto.

In the present invention, in the method of providing information for predicting efficacy or prognosis of cancer immunotherapy or the method for predicting efficacy or prognosis of cancer immunotherapy, the machine learning-based model may be one or more selected from the group consisting of XGBoost and Random forest, but is not limited thereto.

In the present specification, the term “prognosis” refers to a prediction or outlook regarding the future symptoms or course of a disease as determined through diagnosis. In cancer patients, prognosis generally refers to the likelihood of metastasis or the survival period within a certain time after disease onset, surgical procedure, or administration of an immune anticancer agent. For example, it indicates the disease progression and curability, including the likelihood of recurrence, metastatic spread of diseases such as cancer, drug resistance, and cancer-related death or progression. For the purpose of the present invention, the term “prognosis” refers to the possibility of systemic or local recurrence after immune anticancer therapy, and preferably refers to predicting whether systemic or local recurrence will occur within two years after surgical or chemotherapeutic treatment. Prediction of prognosis is a highly important clinical task, as it provides key insights into future cancer treatment strategies, including whether to administer immune anticancer therapy in early-stage cancer patients.

In the present specification, the term “prediction” refers to determining whether a patient preferentially or non-preferentially responds to cancer immunotherapy, and relates to the likelihood and/or possibility of a patient surviving for a certain period after treatment, such as cancer immunotherapy, without cancer recurrence. The prediction method of the present invention may be clinically applied by selecting and administering the most appropriate therapeutic regimen for a particular patient. The prediction method of the present invention may be used to determine whether a patient preferentially responds to a therapeutic regimen, such as administration of an immune anticancer agent, a combination thereof, surgical intervention, or concomitant administration with chemotherapy, or to predict the likelihood of long-term survival or systemic or local recurrence after treatment. In addition, through such prediction, unnecessary cancer immunotherapy may be minimized, and for patients in whom systemic or local recurrence is predicted, more effective adjuvant anticancer therapies may be appropriately planned.

Additionally, the present invention provides a composition for predicting efficacy or prognosis of cancer immunotherapy, comprising an agent for detecting one or more selected from the group consisting of a tumor necrosis factor (TNF) score and immune cell profile.

In the present invention, in the composition for predicting efficacy or prognosis of cancer immunotherapy, the agent is not limited in type as long as it is capable of detecting a tumor necrosis factor (TNF) score or immune cell profile, and all agents used in methods known in the art for detecting a tumor necrosis factor (TNF) score or immune cell profile may be included. For example, the agent may include components, compositions, solutions, or devices conventionally required for single sample gene set enrichment analysis (ssGSEA) based on gene expression using the gene set of the negative regulation of tumor necrosis factor mediated signaling pathway (GO: 0010804) provided by Gene Ontology (http://geneontology.org/) as a method for detecting the tumor necrosis factor (TNF) score, and components, compositions, solutions, or devices conventionally required for ImmuCellAI (Miao, Y. et al., Adv. Sci. 7, 1902880) as a method for detecting the immune cell profile. According to one embodiment of the present invention, the agent for detecting the tumor necrosis factor (TNF) score may comprise a primer or probe that specifically binds to the gene set of the negative regulation of tumor necrosis factor mediated signaling pathway (GO: 0010804), and the agent for detecting the immune cell profile may comprise a primer or probe that specifically binds to one or more marker genes selected from the group consisting of CD8⁺ T cells (CD8⁺ T), central memory T cells (Tcm), cytotoxic T cells (Tc), an immune infiltration score, induced regulatory T cells (iTreg), macrophages, neutrophils, type 1 regulatory T cells (Tr1), type 2 helper T cells (Th2), type 17 helper T cells (Th17), and follicular helper T cells (Tfh), but is not limited thereto.

In the present invention, in the composition for predicting efficacy or prognosis of cancer immunotherapy, the primer or probe is not limited to a specific sequence as long as it is capable of detecting a tumor necrosis factor (TNF) score or an immune cell profile.

Additionally, the present invention provides a kit for predicting efficacy or prognosis of cancer immunotherapy, comprising the composition.

In the kit for predicting efficacy or prognosis of cancer immunotherapy according to the present invention, the term “kit” refers to a tool that allows prediction of efficacy of cancer immunotherapy in a cancer patient by including an agent for detecting a tumor necrosis factor (TNF) score or an immune cell profile. The kit of the present invention may further include, in addition to the agent, other components, compositions, solutions, or devices conventionally required for the measurement or detection methods thereof.

In the kit for predicting efficacy or prognosis of cancer immunotherapy according to the present invention, the kit may comprise a container, an instruction manual, and an agent for detecting a tumor necrosis factor (TNF) score or an immune cell profile. The container may serve to package the agent and may also serve to store and secure it. The material of the container may take the form of, for example, a bottle, tub, sachet, envelope, tube, or ampoule, and may be formed partially or entirely from plastic, glass, paper, foil, wax, or the like. The container may be equipped with a cap that is initially a part of the container or is completely or partially detachable and attachable to the container by mechanical, adhesive, or other means, and may also be equipped with a stopper that allows access to the contents with a syringe needle. The kit may include an external package, and the external package may include instructions for use of the components.

Additionally, the present invention provides a use of a composition comprising an agent for detecting one or more selected from the group consisting of a tumor necrosis factor (TNF) score and immune cell profile for predicting efficacy of cancer immunotherapy.

Additionally, the present invention provides a use of an agent for detecting one or more selected from the group consisting of a tumor necrosis factor (TNF) score and immune cell profile for preparing an agent for predicting efficacy of cancer immunotherapy.

Additionally, the present invention provides a method of providing information for determining or analyzing whether a subject is highly susceptible to prediction of efficacy of cancer immunotherapy, wherein after measuring gene expression from RNA obtained from biological samples collected before and after treatment from a subject suspected of having a high level of efficacy for cancer immunotherapy or a subject suspected of having low efficacy for cancer immunotherapy, and from a control group, when one or more low values selected from the group consisting of a tumor necrosis factor (TNF) score, an immune infiltration score, the abundance score for macrophages, the abundance score for neutrophils, and the abundance score for type 17 helper T cells (Th17) in the immune cell profile are measured, or when high values of the abundance scores for one or more selected from the group consisting of CD8⁺ T cells (CD8⁺ T), central memory T cells (Tcm), cytotoxic T cells (Tc), induced regulatory T cells (iTreg), type 1 regulatory T cells (Tr1), type 2 helper T cells (Th2), and follicular helper T cells (Tfh) in the immune cell profile are measured, it is considered that the subject is one having a high degree of efficacy after cancer immunotherapy or low efficacy for cancer immunotherapy.

The present invention also provides a cancer treatment method comprising:
detecting a tumor necrosis factor (TNF) score and/or an immune cell profile in a biological sample isolated from a subject by measuring a gene expression level, wherein the immune cell profile is one or more selected from the group consisting of CD8⁺ T cells (CD8⁺ T), central memory T cells (Tcm), cytotoxic T cells (Tc), an immune infiltration score, induced regulatory T cells (iTreg), macrophages, neutrophils, type 1 regulatory T cells (Tr1), type 2 helper T cells (Th2), type 17 helper T cells (Th17), and follicular helper T cells (Tfh);
predicting that efficacy or prognosis of cancer immunotherapy is good when one or more gene expression levels selected from the group consisting of a tumor necrosis factor (TNF) score, an immune infiltration score, the abundance score for macrophages, the abundance score for neutrophils, and the abundance score for type 17 helper T cells (Th17) are lower than those of a subject having no efficacy for cancer immunotherapy,
or when the abundance scores for one or more selected from the group consisting of CD8⁺ T cells (CD8⁺ T), central memory T cells (Tcm), cytotoxic T cells (Tc), induced regulatory T cells (iTreg), type 1 regulatory T cells (Tr1), type 2 helper T cells (Th2), and follicular helper T cells (Tfh) are higher than those of a subject having no efficacy for cancer immunotherapy; and
treating the subject predicted to have good efficacy or prognosis for cancer immunotherapy with cancer immunotherapy.

In the present invention, the cancer immunotherapy may comprise administering one or more selected from the group consisting of an agent for immune checkpoint blockade (ICB), an immune cell therapeutic agent, a therapeutic antibody, and an immune enhancer.

In the present invention, when the term “comprising” is used, it indicates that, unless otherwise specified, other components are not excluded and may be further included. Throughout the present specification, the term “step of˜” or “step for˜” does not mean “a step for˜”.

In the present specification, terms of degree such as “about,” “approximately,” “substantially,” and “generally” are used in the sense of being at or near the stated value, taking into account inherent manufacturing and material tolerances, and are employed to prevent unscrupulous infringers from unfairly taking advantage of precise or absolute values disclosed herein for the purpose of understanding the present disclosure. For example, the terms “about,” “approximately,” “substantially,” and “generally” may refer to amounts within less than 10%, less than 5%, less than 3%, less than 1%, less than 0.1%, or less than 0.01% of the stated amount.

In the present specification, the expression “and combinations thereof” included in a Markush-type expression refers to a mixture or combination of one or more selected from the group of components described in the Markush-type expression, and means including one or more selected from the group of such components.

In the present specification, the expression “A and/or B” means “A or B, or both A and B.”

Hereinafter, preferred examples are presented to help understand the present invention. However, the following examples are provided only to help understand the present invention more easily, and the contents of the present invention are not limited by the following examples.

### EXAMPLES

### [Examples for Predicting the Occurrence of Immune-Related Adverse Events Induced by Cancer Immunotherapy]

Example 1. Multicenter Prospective Cohort of Patients with irAEs

A total of 531 patients registered at Seoul Asan Medical Center, consisting of a group of 276 irAE patients that received ICB treatment and a group of 255 non-irAE patients, were enrolled in the study.

The baseline features of the patient cohort, including age, sex, cancer type, and the types of ICB therapies received, are shown in Table 1 below.

TABLE 1

| | irAE group | | non-irAE group | |
|---|---|---|---|---|
| | (N = 276) | | (N = 255) | |
| Age | | | | |
| Median, years (range) | 64.4 ± 9.9 | | 62.7 ± 9.9 | |
| Sex | | | | |
| Male | 214 | (77.5%) | 187 | (73.3%) |
| Female | 62 | (22.5%) | 68 | (26.7%) |
| Cancer type | | | | |
| Gastrointestinal cancer | 96 | (34.8%) | 56 | (22.0%) |
| NSCLC | 94 | (34.1%) | 102 | (40.0%) |
| Urothelial cancer | 41 | (14.9%) | 41 | (16.1%) |
| Pancreatic, hepatic, and | 32 | (11.6%) | 35 | (13.7%) |
| biliary cancer | | | | |
| Melanoma | 5 | (1.8%) | 7 | (2.7%) |
| Breast cancer | 4 | (1.4%) | 1 | (0.4%) |
| Head and neck cancer | 2 | (0.7%) | 9 | (3.5%) |
| Other cancer | 4 | (1.4%) | 1 | (0.4%) |
| Ovary cancer | 0 | (0.0%) | 2 | (0.8%) |
| ICB type* | | | | |
| Nivolumab | 131 | (47.5%) | 93 | (36.5%) |
| Atezolizumab | 75 | (27.2%) | 84 | (32.9%) |
| Pembrolizumab | 38 | (13.8%) | 64 | (25.1%) |
| Nivolumab + Ipilimumab | 14 | (5.1%) | 7 | (2.7%) |
| Durvalumab | 8 | (2.9%) | 4 | (1.6%) |
| Ipilimumab | 1 | (0.4%) | 0 | (0.0%) |
| Investigational Immune | 9 | (3.2%) | 3 | (1.2%) |
| checkpoint treatment | | | | |
| ICB treatment regimen* | | | | |
| ICB monotherapy | 231 | (83.7%) | 215 | (84.3%) |
| ICB + ICB | 11 | (4.0%) | 7 | (2.7%) |
| ICB + cytotoxic | 31 | (11.2%) | 26 | (10.2%) |
| chemotherapy | | | | |
| ICB + molecular targeted | 3 | (1.1%) | 6 | (2.4%) |
| therapy | | | | |
| ICB + cytotoxic chemotherapy + | 0 | (0.0%) | 1 | (0.4%) |
| molecular targeted | | | | |
| therapy | | | | |
| Treatment setting* | | | | |
| Palliative | 258 | (93.5%) | 240 | (94.1%) |
| Maintenance after definitive | 10 | (3.6%) | 11 | (4.3%) |
| CRT | | | | |
| Adjuvant | 6 | (2.2%) | 3 | (1.2%) |
| Neoadjuvant | 2 | (0.7%) | 1 | (0.4%) |
| Prior treatment** | | | | |
| Cytotoxic chemotherapy | 178 | (65.4%) | 160 | (68.7%) |
| Molecular targeted agent | 64 | (24.2%) | 46 | (20.0%) |
| RT | 45 | (16.5%) | 52 | (22.3%) |
| CRT | 37 | (13.6%) | 18 | (7.7%) |
| ECOG performance | | | | |
| 0 | 22 | (8.1%) | 12 | (4.8%) |
| 1 | 221 | (81.0%) | 200 | (80.3%) |
| 2 | 20 | (7.3%) | 22 | (8.8%) |
| >=3 | 10 | (3.6%) | 15 | (6.0%) |

(irAE, immune-related adverse event; NSCLC, non-small cell lung cancer; ICB, immune checkpoint blockade; RT, radiation therapy; CRT, chemoradiotherapy; ECOG performance, performance status)

Example 2. Classification of irAE Patients

For additional analysis and training of the integrated model, 68 types of irAEs (single labels) were classified into 12 major labels (including an ‘Any’ label). The list of single labels composed of each of the 12 major labels is shown in Table 4 of Experimental Example 1 below, and the number of patients for each of the 12 major labels is shown in Table 5 of Experimental Example 1 below.

### Example 3. Clinical Characteristics

Available clinical characteristics for the patients of the cohort of the present invention include drug type, cancer type, ECOG performance status, history of autoimmune disease, and history of diabetes or hypertension. Pretreatment laboratory tests included various combinations calculated from values such as CBC, chemical properties, and neutrophil-to-lymphocyte ratio (NLR), and a platelet-to-lymphocyte ratio (PLR) was also included as a candidate feature.

### Example 4. RNA Sequencing and Data Processing

A library produced from a whole blood sample was sequenced on the Illumina platform with 150 base paired-end reads. The quality of raw FASTQ files was controlled using FastQC (v.0.11.9), MultiQC (v.1.9), and Trimmomatic (v.0.39) (TruSeq3-PE-2.fa: 2:30:10:2: keepBothReads LEADING: 3 TRAILING: 3 MINLEN: 36 for adaptor sequencing trimming), and SortMeRNA (v.2.1b) (silva-euk-18s-id95.fasta, silva-euk-28s-id98.fasta) was used in IRNA filtering.

The reads were aligned on the GRCh38 (hg38) build provided from the 1000 Genomes Project, and genes were assigned based on gencode.v37.annotation.gtf using STAR 2 pass mapping with the --sjdbOverhang 150 option. The aligned reads were classified using SAMtools (v.1.7), and the read count was calculated by HTSeq (v.0.12.4). The read count was normalized by calculating TPM values using in-house code.

Example 5. Deconvolution of Cell Type Abundance from RNA-Seq Data

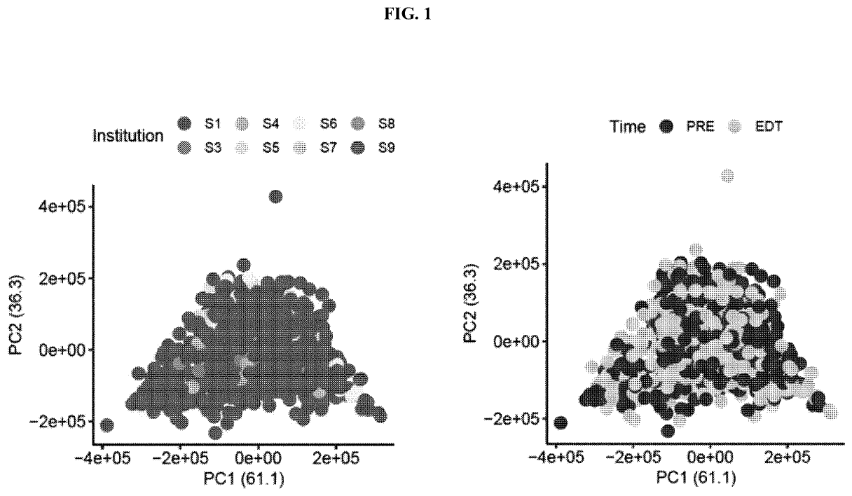

The abundance of a total of 24 immune cells was calculated from whole blood RNA-seq data using ImmuCellAI (Miao, Y et al., Adv. Sci. 7, 1902880.). To minimize potential bias between sequencing data from different sequencing batches, PCA analysis was performed (refer to FIG. 1), and the PCA plot showed that samples from different sequencing batches formed a harmonized mixture.

### Example 6. Assignment of Training and Test Groups

The collected cohort was divided into training groups, used for discovering features associated with irAE occurrence, and test groups, used for model performance validation, at an 8:2 ratio. For unbiased model training and validation, each group was classified based on similar irAE occurrence frequencies.

### Example 7. Statistical Test

To find features exhibiting significant associations with irAE occurrence, the Wilcoxon test was performed immune cell profile. When testing various features, such as immune cell profile, simultaneously, p-value adjustment was performed using the FDR method, and features with an FDR value <0.01 for irAE occurrence were selected as the final features.

### Example 8. Training and Prediction of Integrated Model

The integrated model was trained on irAE occurrence. The features of the integrated model include a neutrophil score, which is closely associated with irAE occurrence as described above, along with immune cell profile including a significant FDR value. For integrated model training, the XGBoost framework and the Random forest framework were implemented. For model training, the patients with irAEs (true cases) and the patients without irAEs (false cases) were divided into training and validation sets at an 8:2 ratio, respectively. All features of the training sets were normalized using StandardScaler, and the scaler suitable for the training sets was applied to transform the features of the validation sets. Since the prediction of irAE occurrence is the primary objective, the optimal model was selected based on the area under the curve (AUC) value, and a test set that was not used for model training was employed for validation.

### Example 9. XGBoost Modeling

The XGBoost framework to which 5-fold cross validation was applied was implemented using the Python XGBoost package. For hyperparameter tuning, the gridSearchCV algorithm was used, and the following parameter sets were used (n_estimators: [50, 100, 200, 500, 1000], learning_rate: [0.01, 0.05, 0.1, 0.3], max_depth: [3, 4, 5, 6, 7, 8, 9, 10], subsample: [0.5, 0.7, 0.8, 0.9, 1.0], colsample_bytree: [0.5, 0.7, 0.8, 0.9, 1.0], gamma: [0, 0.1, 0.2, 0.3, 0.4], min_child_weight: [1, 3, 5, 7]).

### Example 10. Random Forest Modeling

The Random forest framework to which 5-fold cross validation was applied was implemented using the Python RandomForestClassifier package. For hyperparameter tuning, the gridSearchCV algorithm was used, and the following parameter sets were used (n_estimators: [50, 100, 200, 500, 1000], max_depth: [3, 4, 5, 6, 7, 8, 9, 10], min_samples_split: [2, 5, 10], min_samples_leaf: [1, 2, 4], bootstrap: [True, False]).

### Example 11. Evaluation of Feature Importance

A feature importance value, used to interpret the impact of each feature on the prediction result, was calculated using the feature_importances_function of the XGBoost package. For relative comparison of feature importance, the importance of each feature was expressed as the value divided by the highest feature importance score.

### [Examples for Predicting Efficacy or Prognosis of Cancer Immunotherapy]

Example 12. Multicenter Prospective Cohort of Patient Treated with Cancer Immunotherapy

A group of 182 patients registered at Seoul Asan Medical Center, who received ICB treatment and were evaluated for therapeutic responses according to RECIST 1.1, which is efficacy Evaluation Criteria in Solid Tumors, were enrolled in the study. In the present invention, the clinical response group (DCB) refers to patients who exhibit a complete response (CR) or partial response (PR) according to the RECIST 1.1 criteria, as well as those with stable disease (SD) showing a progression free survival (PFS) of 6 months or longer or an overall survival (OS) of 1 year or longer, whereas the non-clinical benefit group (NCB) refers to all other patients. In addition, blood samples were collected from each patient both immediately before ICB treatment (PRE) and within 2 to 8 weeks after the initial administration of ICB treatment (EDT).

The baseline features of the patient cohort, including age, sex, cancer type, and the type of ICB agent administered are shown in Table 2 below.

TABLE 2

| | |
|---|---|
| DCB group | NCB group |
| (N = 38) | (N = 144) |

Age

| | | |
|---|---|---|
| Median, years (range) | 63.7 ± 9.5 | 61.9 ± 9.4 |

| | | | | |
|---|---|---|---|---|
| Sex | | | | |
| Male | 33 | (86.8%) | 112 | (77.8%) |
| Female | 5 | (13.2%) | 32 | (22.2%) |
| Cancer type | | | | |
| NSCLC | 18 | (47.4%) | 50 | (34.7%) |
| Pancreatic, hepatic, and biliary | 6 | (15.8%) | 22 | (15.3%) |
| cancer | | | | |
| Urothelial cancer | 6 | (15.8%) | 21 | (14.6%) |
| Gastrointestinal cancer | 4 | (10.5%) | 47 | (32.6%) |
| Melanoma | 2 | (5.3%) | 1 | (0.7%) |
| Breast cancer | 1 | (2.6%) | 0 | (0.0%) |
| Ovary cancer | 1 | (2.6%) | 0 | (0.0%) |
| Head and neck cancer | 0 | (0.0%) | 2 | (1.4%) |
| Other cancer | 0 | (0.0%) | 1 | (0.7%) |
| ICB type | | | | |
| Nivolumab | 15 | (39.5%) | 74 | (51.4%) |
| Atezolizumab | 9 | (23.7%) | 43 | (29.9%) |
| Pembrolizumab | 9 | (23.7%) | 17 | (11.8%) |
| Nivolumab + Ipilimumab | 1 | (2.6%) | 4 | (2.8%) |
| Durvalumab | 1 | (2.6%) | 0 | (0.0%) |
| Tislelizumab | 0 | (0.0%) | 1 | (0.7%) |
| Investigational Immune | 3 | (7.9%) | 5 | (3.5%) |
| checkpoint treatment | | | | |
| ICB treatment regimen | | | | |
| ICB monotherapy | 35 | (92.1%) | 139 | (96.5%) |
| ICB + chemotherapy | 2 | (5.3%) | 1 | (0.7%) |
| ICB + ICB | 1 | (2.6%) | 4 | (2.8%) |
| ECOG performance | | | | |
| 0 | 2 | (5.3%) | 5 | (3.5%) |
| 1 | 33 | (86.8%) | 123 | (85.4%) |
| >=2 | 3 | (7.9%) | 16 | (11.1%) |

(NSCLC, non-small cell lung cancer; ICB, immune checkpoint blockade; ECOG performance, performance status; Other cancer, Thymic carcinoma; Investigational Immune checkpoint treatment, PDR001 or Tislelizumab)

### Example 13. Clinical Features

The available clinical features for patients in the cohort of the present invention include drug type, cancer type, ECOG performance status, history of autoimmune disease, and history of diabetes or hypertension. Pretreatment laboratory tests include various combinations calculated from values such as complete blood court (CBC), chemical properties, neutrophil-to-lymphocyte ratio (NLR), and platelet-to-lymphocyte ratio (PLR).

### Example 14. RNA Sequencing and Data Treatment

A library produced from the whole blood sample was sequenced on the Illumina platform with 150 base paired-end reads. The quality of raw FASTQ files was controlled using FastQC (v.0.11.9), MultiQC (v.1.9), and Trimmomatic (v.0.39) (TruSeq3-PE-2.fa: 2:30:10:2: keepBothReads LEADING: 3 TRAILING: 3 MINLEN: 36 for adaptor sequencing trimming), and SortMeRNA (v.2.1b) (silva-euk-18s-id95.fasta, silva-euk-28s-id98.fasta) was used in rRNA filtering.

The reads were aligned on the GRCh38 (hg38) build provided from the 1000 Genomes Project, and genes were assigned based on gencode.v37.annotation.gtf using STAR 2 pass mapping with the --sjdbOverhang 150 option. The aligned reads were classified using SAMtools (v.1.7), and the read count was calculated by HTSeq (v.0.12.4). The read count was normalized by calculating TPM values using in-house code.

Example 15. Deconvolution of Cell Type Abundance from RNA-Seg Data

The abundance of a total of 25 immune cells was calculated from whole blood RNA-seq data using an immune cell abundance identifier (ImmuCellAI) (Miao, Y et al., Adv. Sci. 7, 1902880.). To minimize potential biases between sequencing data from different sequencing batches, principal component analysis (PCA) was performed, and the resulting PCA plot showed that samples from different sequencing batches formed a harmonized mixture.

### Example 16. Assignment of Training and Test Groups

The collected cohort was divided into training groups, used for discovering features associated with efficacy to an immune anticancer agent, and test groups, used for model performance validation, at an 8:2 ratio. For unbiased model training and validation, each group was classified based on the frequency of efficacy to an immune anticancer agent.

### Example 17. Statistical Test

To find features exhibiting significant associations with efficacy to an immune anticancer agent, the Wilcoxon test was performed. When testing various features, such as immune cell profile, simultaneously, p-value adjustment was performed using the FDR method, and features with an FDR value <0.05 for efficacy were selected as the final features.

### Example 18. Training and Prediction of Integrated Model

The integrated model was trained to predict efficacy to an immune anticancer agent. The features of the integrated model include a tumor necrosis factor (TNF) score, which is closely associated with efficacy to an immune anticancer agent as described above, along with immune cell profile including a significant FDR value. For integrated model training, the XGBoost framework and the Random forest framework were implemented. For model training, patients exhibiting efficacy to the immune anticancer agent (true cases) and patients exhibiting no efficacy to the immune anticancer agent (false cases) were divided into training and validation sets at a ratio of 8:2, respectively. All features of the training sets were normalized using MinmaxScaler, and the scaler suitable for the training sets was applied to transform the features of the validation sets. Since the prediction of efficacy to an immune anticancer agent is the primary objective, the optimal model was selected based on the area under the curve (AUC) value, and a test set that was not used for model training was employed for validation.

### Example 19. XGBoost Modeling

The XGBoost framework to which 5-fold cross validation was applied was implemented using the Python XGBoost package. For hyperparameter tuning, the gridSearchCV algorithm was used, and the following parameter sets were used (n_estimators: [50, 100, 200, 500, 1000], learning_rate: [0.01, 0.05, 0.1, 0.3], max_depth: [3, 4, 5, 6, 7, 8, 9, 10], subsample: [0.5, 0.7, 0.8, 0.9, 1.0], colsample_bytree: [0.5, 0.7, 0.8, 0.9, 1.0], gamma: [0, 0.1, 0.2, 0.3, 0.4], min_child_weight: [1, 3, 5, 7]).

### Example 20. Random Forest Modeling

The Random forest framework to which 5-fold cross validation was applied was implemented using the Python RandomForestClassifier package. For hyperparameter tuning, the gridSearchCV algorithm was used, and the following parameter sets were used (n_estimators: [50, 100, 200, 500, 1000], max_depth: [3, 4, 5, 6, 7, 8, 9, 10], min_samples_split: [2, 5, 10], min_samples_leaf: [1, 2, 4], bootstrap: [True, False]).

### Example 21. Evaluation of Feature Importance

A feature importance value, used to interpret the impact of each feature on the prediction result, was calculated using the feature_importances_function of the XGBoost package. For relative comparison of feature importance, the importance of each feature was expressed as the value divided by the highest feature importance score.

### Experimental Examples

### [Experimental Examples for Predicting the Occurrence of Immune-Related Adverse Events Induced by Cancer Immunotherapy]

Experimental Example 1. Multicenter Pan-Cancer Prospective Cohort of irAEs

Among 531 patients treated with ICB, 276 patients were identified to have 68 types of irAEs (refer to Table 3 below). Labels were assigned to each irAE based on the affected organ systems, such as skin, endocrine system, thyroid gland, musculoskeletal system, gastrointestinal system, and neurological system. Patients with 3 or more types of irAEs of any severity grade were labeled as Multiple G>=1, patients with 3 or more types of irAEs of grade 2 or higher were labeled as Multiple G>=2, and patients with any irAE of grade 3 or higher as well as those with critical irAEs of grade 2 or higher were additionally labeled as Critical. Other labels include Flu-like (flu-like symptoms) and Pulmonary (pneumonia caused by ICB treatment), and patients in all irAE categories were labeled as Any (refer to Table 4 below).

TABLE 3

| | | irAE of | irAE of non- |
|---|---|---|---|
| Type of irAE | Control | interest | interest |
| Pruritus | 255 | 111 | 165 |
| Skin rash | 255 | 87 | 189 |
| Fatigue | 255 | 45 | 231 |
| Myalgia | 255 | 41 | 235 |
| Hypothyroidism | 255 | 32 | 244 |
| Pneumonitis | 255 | 24 | 252 |
| Arthralgia | 255 | 17 | 259 |
| Amylase | 255 | 16 | 260 |
| Lipase | 255 | 16 | 260 |
| Enterocolitis | 255 | 15 | 261 |
| Headache | 255 | 15 | 261 |
| Dry mouth | 255 | 13 | 263 |
| Fever | 255 | 13 | 263 |
| Nausea | 255 | 12 | 264 |
| Anorexia | 255 | 10 | 266 |
| Liver enzyme | 255 | 10 | 266 |
| Hyperthyroidism | 255 | 9 | 267 |
| Neuropathy | 255 | 9 | 267 |
| Sweating | 255 | 9 | 267 |
| Adrenal insufficiency | 255 | 7 | 269 |
| Dizziness | 255 | 7 | 269 |
| Edema facial | 255 | 6 | 270 |
| Urticaria | 255 | 5 | 271 |
| Infusion chills | 255 | 5 | 271 |
| Infusion | 255 | 5 | 271 |
| Dermatitis | 255 | 4 | 272 |
| Abdominal pain | 255 | 4 | 272 |
| Vomiting | 255 | 4 | 272 |
| Muscle weakness | 255 | 4 | 272 |
| Creatine phosphokinase elevation | 255 | 4 | 272 |
| Dry eye | 255 | 4 | 272 |
| Lichen planus | 255 | 3 | 273 |
| Pancreatitis | 255 | 3 | 273 |
| Constipation | 255 | 3 | 273 |
| Stomatitis | 255 | 3 | 273 |
| Type I diabetes mellitus | 255 | 3 | 273 |
| Edema limb | 255 | 3 | 273 |
| Hand foot syndrome | 255 | 2 | 274 |
| Paronychia | 255 | 2 | 274 |
| Dysesthesia | 255 | 2 | 274 |
| Cognitive dysfunction | 255 | 2 | 274 |
| Pericarditis | 255 | 2 | 274 |
| Hoarseness | 255 | 2 | 274 |
| Infusion chest | 255 | 2 | 274 |
| Skin hypopigmentation | 255 | 1 | 275 |
| Dry skin | 255 | 1 | 275 |
| Bullous pemphigoid | 255 | 1 | 275 |
| Myositis | 255 | 1 | 275 |
| Exacerbation of rheumatoid | 255 | 1 | 275 |
| arthritis | | | |
| Myopathy | 255 | 1 | 275 |
| Meningoencephalitis | 255 | 1 | 275 |
| Eosinophilia | 255 | 1 | 275 |
| Nephritis | 255 | 1 | 275 |
| Creatinine increased | 255 | 1 | 275 |
| Proteinuria | 255 | 1 | 275 |
| Hematuria | 255 | 1 | 275 |
| Renal thrombotic microangiopathy | 255 | 1 | 275 |
| Blurred vision | 255 | 1 | 275 |
| Retinopathy | 255 | 1 | 275 |
| Tinnitus | 255 | 1 | 275 |
| Hyposmia | 255 | 1 | 275 |
| Sore throat | 255 | 1 | 275 |
| Insomnia | 255 | 1 | 275 |
| Herpes zoster | 255 | 1 | 275 |
| Infusion throat | 255 | 1 | 275 |
| Hypercalcemia | 255 | 1 | 275 |
| Eczema | 255 | 1 | 275 |
| Acute tubular necrosis | 255 | 1 | 275 |

TABLE 4

| Type of irAE | Description | Components |
|---|---|---|
| Any | Any kinds of irAE | single labels |
| Critical | Any irAEs of grade ≥3 or | *adrenal insufficiency, type I diabetes mellitus, |
| | critical irAEs* of grade ≥2 | pancreatitis, meningoencephalitis, pericarditis, nephritis, |
| | | renal thrombotic microangiopathy, pneumonitis |
| Skin | irAEs related with skin | skin hypopigmentation |
| | | hand foot syndrome |
| | | dry skin |
| | | dermatitis |
| | | skin rash |
| | | paronychia |
| | | lichen planus |
| | | pruritus |
| | | urticaria |
| | | bullous pemphigoid |
| Thyroid | irAEs related with thyroid gland | hyperthyroidism |
| | | hypothyroidism |
| | | subclinical hypothyroidism with TSH >10 μU/mL |
| Endocrine | irAEs related with endocrine | hyperthyroidism |
| | system | hypothyroidism |
| | | subclinical hyperthyroidism |
| | | subclinical hypothyroidism |
| | | subclinical hypothyroidism with TSH >10 μU/mL |
| | | adrenal insufficiency |
| | | type I diabetes mellitus |
| Musculoskeletal | irAEs related with | arthralgia |
| | musculoskeletal system | myalgia |
| | | myositis |
| | | myopathy |
| | | muscle weakness |
| | | exacerbation of rheumatoid arthritis |
| Neurologic | irAEs related with neurologic | meningoencephalitis |
| | system | headache |
| | | dizziness |
| | | cognitive dysfunction |
| | | dysesthesia |
| Pulmonary | Pneumonitis of any grade | pneumonitis |
| Gastrointestinal | irAEs related with | pancreatitis |
| | gastrointestinal system | asymptomatic amylase/lipase elevation |
| | | enterocolitis |
| | | nausea |
| | | vomiting |
| | | anorexia |
| | | abdominal pain |
| | | constipation |
| Flu-like | Flu-like symptoms | fever |
| | | sweating |
| | | fatigue |
| | | arthralgia |
| | | muscle weakness |
| | | myalgia |
| | | sore throat |
| | | headache |
| Multiple (any | Patients with 3 or more irAEs of | single labels |
| grade) | any grade | |
| Multiple | Patients with 3 or more irAEs of | single labels |
| (grade ≥2) | grade ≥2 | |

Referring to Table 5 below, 52% of all cases were labeled as Any, whereas skin was the most frequent irAE type (26%), followed by flu-like (21%) and multiple (all 5 grades) (18%).

TABLE 5

| | | irAE of | irAE of non- |
|---|---|---|---|
| Type of irAE | Control | interest | interest |
| Any | 255 | 276 | 0 |
| Skin | 255 | 140 | 136 |
| Flu-like | 255 | 110 | 166 |
| Multiple (any grade) | 255 | 95 | 181 |
| Endocrine | 255 | 83 | 193 |
| Gastrointestinal | 255 | 65 | 211 |
| Musculoskeletal | 255 | 58 | 218 |
| Critical | 255 | 49 | 227 |
| Thyroid | 255 | 46 | 230 |
| Multiple (grade >=2) | 255 | 44 | 232 |
| Neurologic | 255 | 33 | 243 |
| Pulmonary | 255 | 24 | 252 |

In addition, to identify genetic, molecular, and cellular risk factors of irAEs, multidimensional sequencing was performed on this cohort. RNA sequencing was performed on 531 matched whole blood samples before ICB treatment (hereinafter, PRE) or early during treatment (EDT) of ICB treatment to investigate differential molecular activity and immune cell profiles between patients with and without irAEs and between PRE and EDT.

Experimental Example 2. Identification of irAE-Associated Neutrophil and Immune Cell Profile in Both PRE and EDT irAE Samples

Gene expression was analyzed through RNA sequencing for the 531 matched whole blood samples at PRE and EDT of ICB treatment. Principal component analysis was performed to determine whether there was a batch effect by the organ of origin and the time of blood collection. As a result, as shown in FIG. 1, it was confirmed that all samples were well integrated regardless of the organ of origin and the time of blood collection.

For model construction, all samples were classified into training sets and test sets at an 8:2 ratio based on the incidence of irAEs (number of training sets=424, number of test sets=107). To identify the comprehensive characteristics of neutrophils, a neutrophil score was calculated based on gene expression. The neutrophil score was calculated by quantification and normalization through Single Sample Gene Set Enrichment Analysis using the neutrophil mediated immunity pathway gene set provided from Gene Ontology (http://geneontology.org/) based on gene expression.

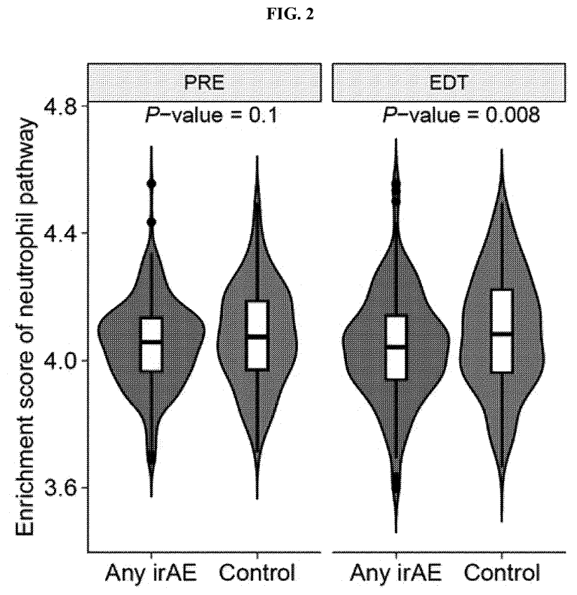

The correlation between the neutrophil score and irAE occurrence in the training set showed that, as shown in FIG. 2, patients with irAEs exhibited a lower neutrophil score, and this trend was more evident in EDT samples.

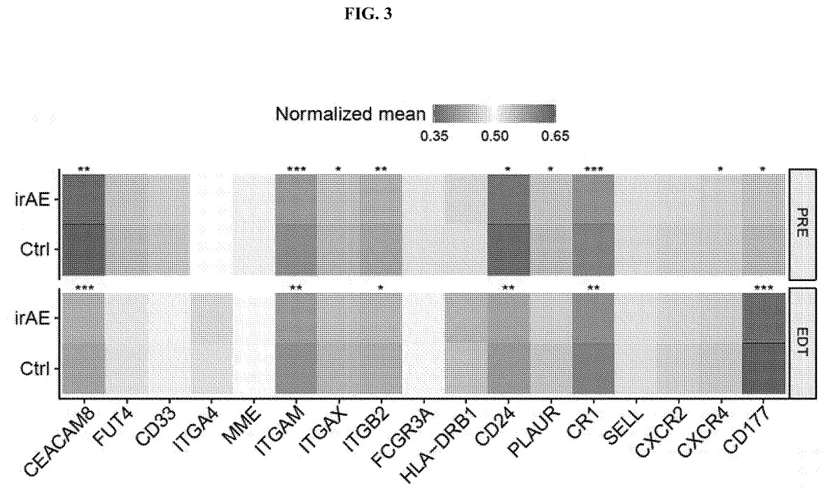

In addition, apart from the confirmation of the expression of neutrophil-mediated immunity pathway gene set to confirm the neutrophil score, the expression levels of neutrophil marker genes were determined. As a result, as shown in FIG. 3, in both PRE and EDT samples, the expression levels were significantly lower in the irAE group than in the control (irAE=137 subjects, and control=122 subjects).

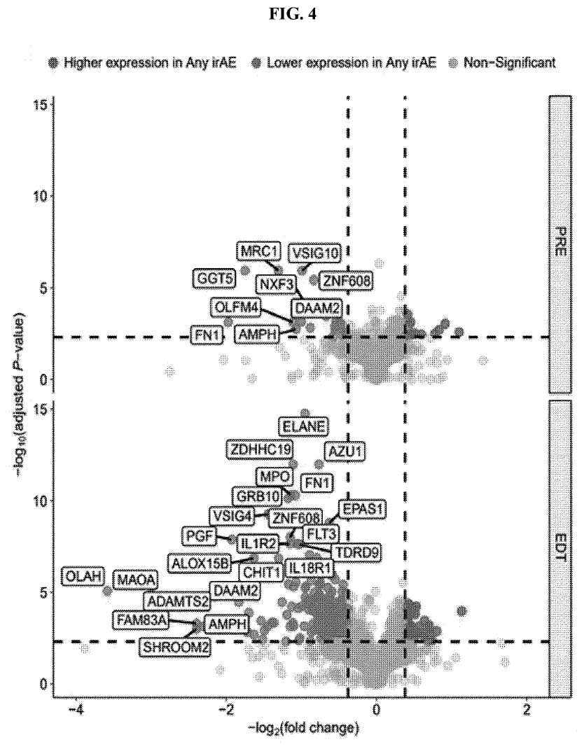

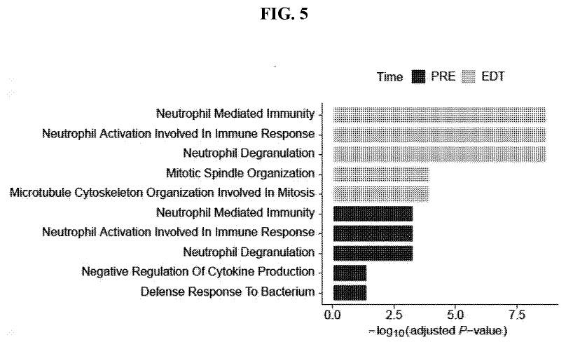

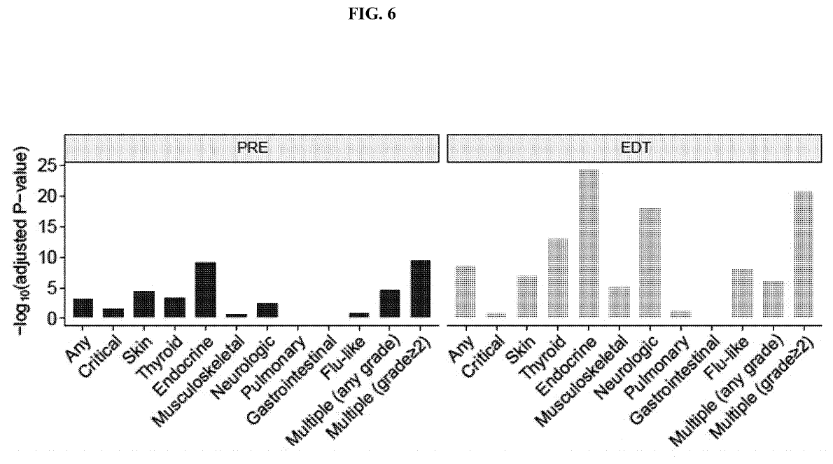

Afterward, irAE-related number differences were characterized at the molecular level beyond the number of cells. To this end, genes more highly expressed or underexpressed in the irAE samples were identified. As a result, as shown in FIG. 4, particularly, differential gene expression was characterized by underexpression mostly in the irAE group, which was more clearly seen in the EDT samples than in the PRE samples. Pathway enrichment analysis showed that, as shown in FIG. 5, irAE-related gene suppression occurred not only in neutrophil-mediated immunity but also in neutrophil activation and degranulation in both PRE and EDT samples. However, the degree of pathway enrichment was much greater for the genes identified in the EDT samples. When each of 12 irAE groups was identified, a similar pattern as observed when underexpressed genes were identified from a comparison between each of the 12 irAE groups and the control, as shown in FIG. 6.

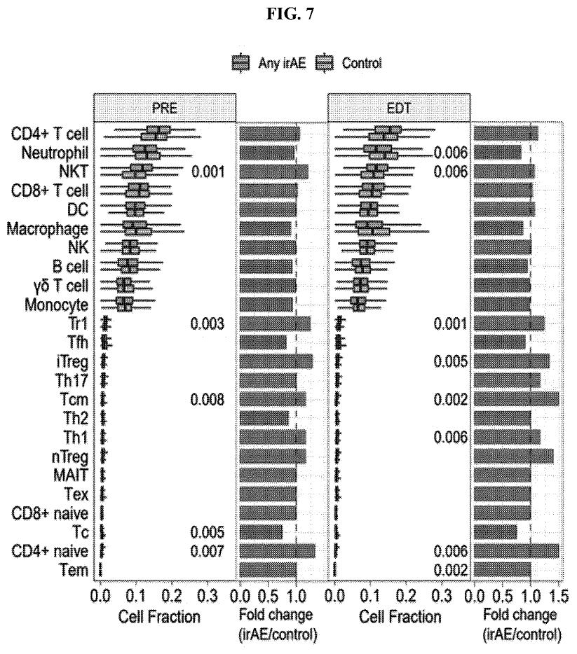

Afterward, apart from the confirmation of the neutrophil score and the neutrophil marker gene expression level, the association with irAEs at the immune cell level was determined in the training set. As a result, as shown in FIG. 7, immune cell types exhibiting a significant association with irAE occurrence in both cases of PRE and EDT were identified, and this trend was more pronounced in the EDT samples (upper bar: irAE, lower bar: control).

Experimental Example 3. Establishment and Evaluation of RNA-Based irAE Prediction Model

As confirmed in Experimental Example 2 above, the features associated with irAEs in the training sets for the PRE and EDT samples showed that this trend was more pronounced in the EDT samples. Based on this, an irAE occurrence prediction model was established using 10 features (FDR value <0.01) of the neutrophil score and immune cell profile (neutrophils, natural killer T cells (NKT), type 1 regulatory T cells (Tr1), type 1 helper T cells (Th1), induced regulatory T cells (iTreg), central memory T cells (Tcm), naive CD4⁺ T cells, effector memory T cells (Tem), and cytotoxic T cells (Tc)), with a significant association with irAE occurrence in the PRE and EDT samples.

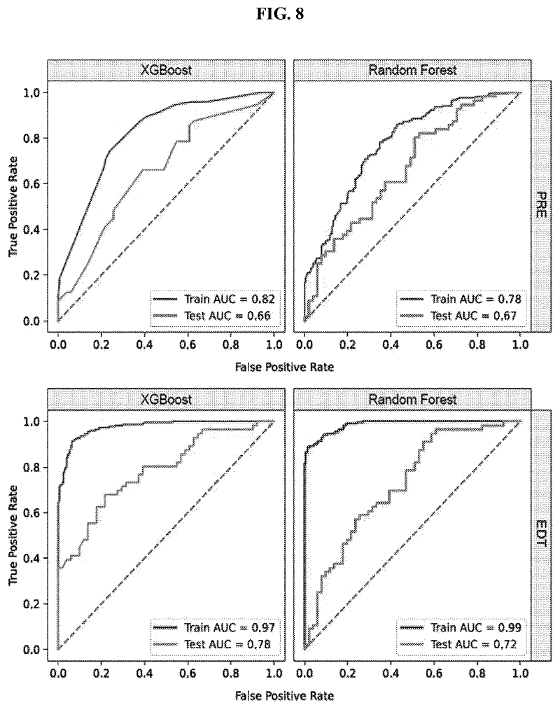

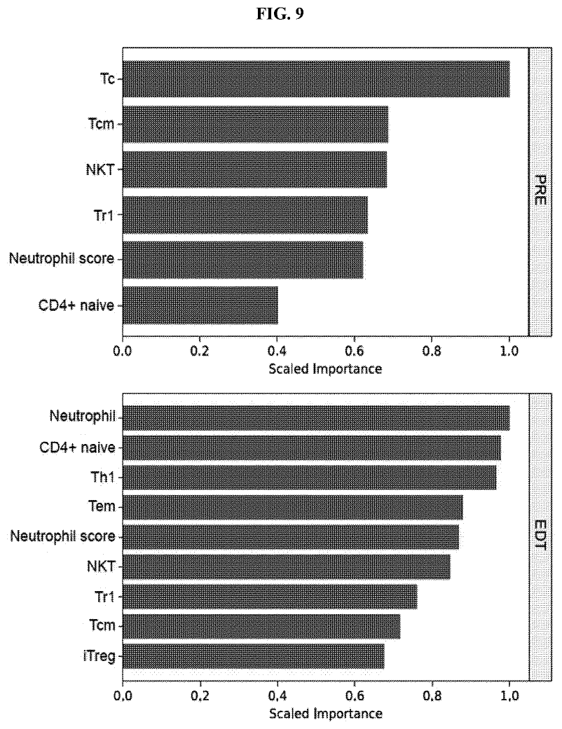

As a result of verifying the model trained with the training set on the test set, as shown in FIG. 8, in the case of PRE, the test AUC value of the XGBoost model was 0.66, and the test AUC value of the Random forest model was 0.67, whereas, in the case of EDT, the test AUC value of the XGBoost model was 0.78, and the test AUC value of the Random forest model was 0.72. Therefore, it can be seen that the XGBoost model exhibited much better performance than the Random forest model. In addition, when the importance of the 10 features was confirmed in the XGBoost model, as shown in FIG. 9, for PRE, Tc was identified as the most important feature, whereas for EDT, the neutrophil was identified as the most important feature.

### [Experimental Examples for Predicting Efficacy or Prognosis of Cancer Immunotherapy]

### Experimental Example 4. Multicenter Pan-Cancer Prospective Cohort for Cancer Immunotherapy

Efficacy was confirmed in 38 of 182 patients treated with ICB, but not confirmed in 144 patients.

In addition, to identify genetic, molecular, and cellular factors, multidimensional sequencing was performed on this cohort. RNA sequencing was performed on the 182 matched whole blood samples at PRE and EDT of ICB treatment to investigate differential molecular activity and immune cell profiles between patients with and without efficacy and between PRE and EDT.

### Experimental Example 5. Confirmation of Efficacy-Associated Signaling Pathway Enrichment and Immune Cell Profiles in PRE and EDT Samples

Gene expression was analyzed through RNA sequencing for the 182 matched whole blood samples at PRE and EDT of ICB treatment. The correlation between efficacy and each of gene expression, immune cell profile, immune-related signaling pathway enrichment, and immune repertoire was evaluated.

For model construction, all samples were classified into training sets and test sets at an 8:2 ratio based on efficacy ratio (number of training sets=145, number of test sets=37). To identify comprehensive characteristics, TNF scores, and the expression levels of neutrophil marker genes and immune cell marker genes were calculated based on gene expression. The tumor necrosis factor score was calculated by quantification and normalization through Single Sample Gene Set Enrichment Analysis using the negative regulation of tumor necrosis factor-mediated signaling pathway (GO: 0010804) gene set provided from Gene Ontology (http://geneontology.org/) based on gene expression.

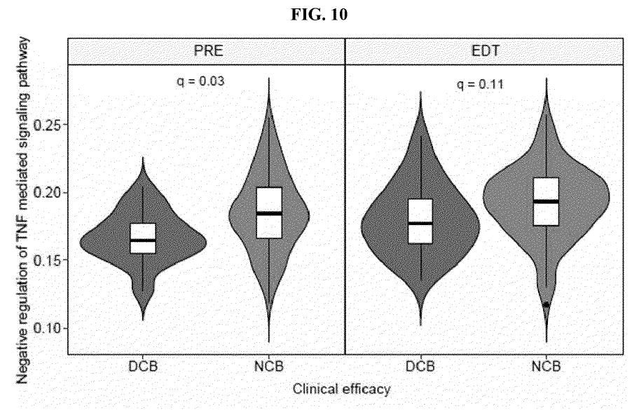

As a result of confirming the correlation between the TNF score and efficacy in the training set, as shown in FIG. 10, a lower TNF score was observed in patients with efficacy, and this trend was more evident in the PRE samples.

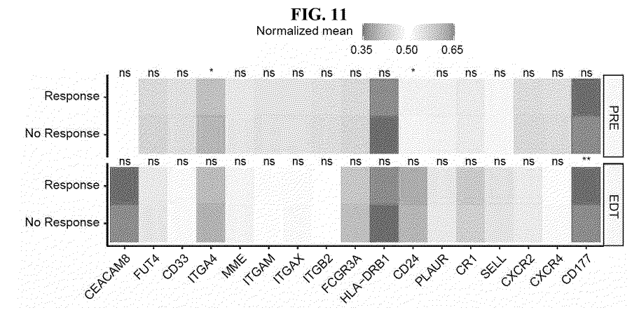

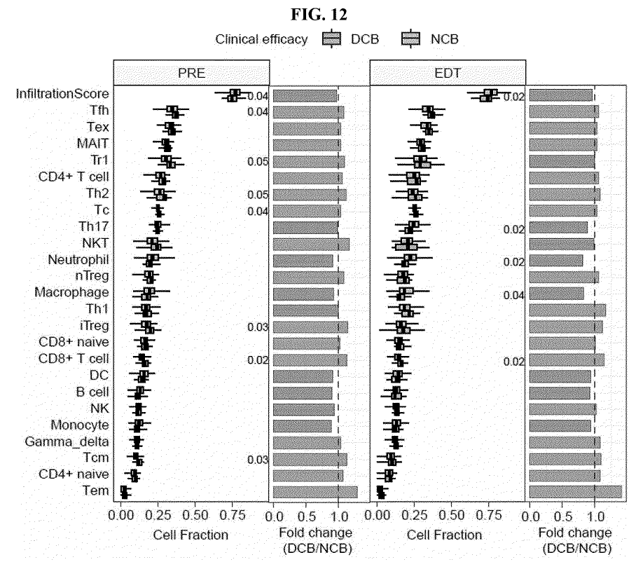

The correlation of efficacy with the expression of neutrophil surface marker genes, known to be associated with irAEs in ICB treatment was examined in the training set, which confirmed that, as shown in FIG. 11, neutrophil surface marker genes were not generally associated with efficacy. In addition, as a result of examining the correlation of efficacy with immune cell profile, analyzed using ImmuCellAI, in the training set, as shown in FIG. 12, it was confirmed that CD8⁺ T cells (CD8⁺ T), central memory T cells (Tcm), cytotoxic T cells (Tc), immune infiltration scores, induced regulatory T cells (iTreg), macrophages (Macrophage), neutrophil (Neutrophil), type 1 regulatory T cells (Tr1), type 2 helper T cells (Th2), type 17 helper T cells (Th17), and follicular helper T cells (Tfh) are associated with efficacy for cancer immunotherapy.

### Experimental Example 6. Establishment and Evaluation of RNA-Based Efficacy Prediction Model

As a result of confirming features associated with efficacy in the training sets for the PRE and EDT samples as confirmed in Experimental Example 5, immune-related signaling pathway enrichment and efficacy-associated features in the immune cell profile were able to be confirmed (FIGS. 10 and 12). Based on this, a efficacy prediction model was established using 12 features (FDR value <0.05) of the TNF score and the immune cell profile (CD8⁺ T cells (CD8⁺ T), central memory T cells (Tcm), cytotoxic T cells (Tc), immune infiltration score, induced regulatory T cells (iTreg), macrophages (Macrophage), neutrophil (Neutrophil), type 1 regulatory T cells (Tr1), type 2 helper T cells (Th2), type 17 helper T cells (Th17), and follicular helper T cells (Tfh)), with a significant association with efficacy in the PRE and EDT samples.

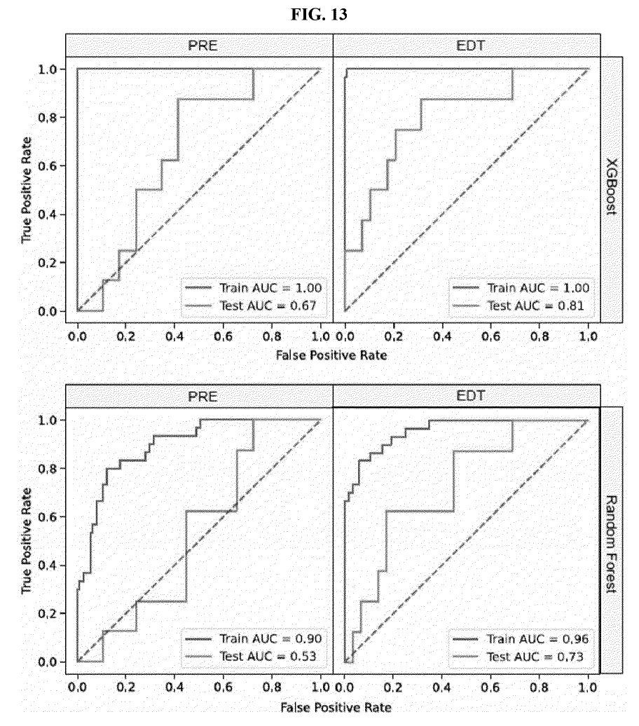

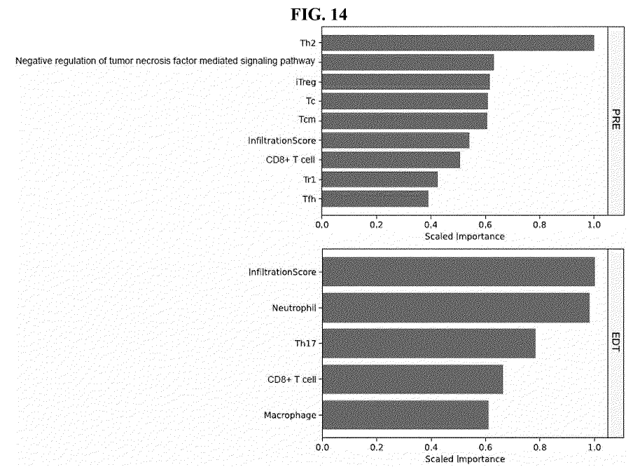

As a result of verifying the model trained with the training sets, as shown in FIG. 13, in the case of PRE, the test AUC value of the XGBoost model was 0.67, and the test AUC value of the Random forest model was 0.53, whereas, in the case of EDT, the test AUC value of the XGBoost model was 0.81, and the test AUC value of the Random forest model was 0.73. Therefore, it can be seen that the XGBoost model exhibits much better performance than the Random forest model. In addition, when the importance of the 12 features was verified in the XGBoost model, as shown in FIG. 14, for PRE, type 2 helper T cells (Th2) were identified as the most important feature, whereas for EDT, the immune infiltration score was identified as the most important feature.

The foregoing description of the present invention is for illustrative purposes only, and it will be understood by those skilled in the art that various modifications can be made in other specific forms without departing from the technical spirit or essential characteristics of the present invention. Therefore, the embodiments described above are to be understood as illustrative and not restrictive in every respect.

### INDUSTRIAL APPLICABILITY

The neutrophil score or immune cell profile according to the present invention is expected to be useful as a biomarker for predicting the occurrence of an immune-related adverse event induced by cancer immunotherapy or for predicting efficacy of cancer immunotherapy, and the gene expression-based tumor necrosis factor (TNF) score or immune cell profile according to the present invention is expected to be useful as a biomarker for predicting efficacy or prognosis of cancer immunotherapy, and thus has industrial applicability.

1. An analytical method for determining whether a subject receiving cancer immunotherapy has susceptibility or resistance to the occurrence of an immune-related adverse event (irAE) induced by cancer immunotherapy, comprising measuring the expression of a gene in a biological sample isolated from the subject; and detecting one or more selected from the group consisting of a neutrophil score and immune cell profile.

2. The analytical method of claim 1, wherein the expression of a gene is measured by one or more methods selected from the group consisting of sequencing, RNA sequencing, and next generation sequencing (NGS).

3. The analytical method of claim 1, wherein the immune cell profile comprises a set of abundance scores for one or more selected from the group consisting of neutrophils, natural killer T cells (NKT), type 1 regulatory T cells (Tr1), type 1 helper T cells (Th1), induced regulatory T cells (iTreg), central memory T cells (Tcm), naive CD4⁺ T cells, effector memory T cells (Tem), and cytotoxic T cells (Tc).

4. The analytical method of claim 1, wherein the subject is a cancer patient before or after treatment with cancer immunotherapy.

5. The analytical method of claim 1, wherein the biological sample is one or more selected from the group consisting of tissue, cells, whole blood, serum, plasma, saliva, sputum, cerebrospinal fluid, urine, and stool, isolated from the subject.

6. The analytical method of claim 1, wherein the immune-related adverse event is one or more selected from the group consisting of a skin adverse event, an endocrine system adverse event, a thyroid gland adverse event, a musculoskeletal system adverse event, a gastrointestinal system adverse event, a neurological system adverse event, a flu-like symptom, and a pulmonary symptom, which occur due to cancer immunotherapy.

7. The analytical method of claim 1, further comprising:
predicting that the risk of occurrence of immune-related adverse events is high, when one or more selected from the group consisting of a neutrophil score, an abundance score for neutrophils in the immune cell profile, and an abundance score for cytotoxic T cells in the immune cell profile are lower in the biological sample isolated from the subject compared to a control, or
predicting that the risk of occurrence of immune-related adverse events is high, when abundance scores for one or more selected from the group consisting of natural killer T cells (NKT), type 1 regulatory T cells (Tr1), type 1 helper T cells (Th1), induced regulatory T cells (iTreg), central memory T cells (Tcm), naive CD4⁺ T cells, and effector memory T cells (Tem) in the immune cell profile are higher in the biological sample isolated from the subject compared to a control.

8. The analytical method of claim 1, further comprising:
predicting the risk of occurrence of an immune-related adverse event induced by cancer immunotherapy through a machine learning-based model by detecting one or more selected from the group consisting of a neutrophil score and immune cell profile.

9. The analytical method of claim 8, wherein the machine learning-based model is one or more selected from the group consisting of XGBoost and Random forest.

10. A method for treating cancer, comprising:
measuring gene expression in a biological sample isolated from a subject;
detecting one or more selected from the group consisting of a neutrophil score and immune cell profile;
predicting that the risk of developing an immune-related adverse event is low when one or more selected from the group consisting of a neutrophil score, an abundance score for neutrophils in the immune cell profile, and an abundance score for cytotoxic T cells in the immune cell profile are higher than those of a subject who developed an immune-related adverse event induced by cancer immunotherapy in the biological sample isolated from the subject, or when the abundance scores for one or more selected from the group consisting of natural killer T cells (NKT), type 1 regulatory T cells (Tr1), type 1 helper T cells (Th1), induced regulatory T cells (iTreg), central memory T cells (Tcm), naive CD4⁺ T cells, and effector memory T cells (Tem) in the immune cell profile are lower than those of a subject who developed an immune-related adverse event induced by cancer immunotherapy in the biological sample isolated from the subject; and
treating the subject predicted to have a low risk of developing the immune-related adverse event with cancer immunotherapy.

11. The method of claim 10, wherein the cancer immunotherapy comprises administering one or more selected from the group consisting of an agent for immune checkpoint blockade (ICB), an immune cell therapeutic agent, a therapeutic antibody, and an immune enhancer.

12. An analytical method for determining whether a subject receiving cancer immunotherapy has susceptibility or resistance to cancer immunotherapy, comprising measuring the expression of a gene in a biological sample isolated from the subject; and detecting one or more selected from the group consisting of a neutrophil score and immune cell profile.

13. An analytical method for determining whether a subject receiving cancer immunotherapy has susceptibility or resistance to cancer immunotherapy, comprising:
detecting a tumor necrosis factor (TNF) score and/or immune cell profile in a biological sample isolated from the subject by measuring a gene expression level,
wherein the immune cell profile comprises one or more selected from the group consisting of CD8⁺ T cells, central memory T cells (Tcm), cytotoxic T cells (Tc), an immune infiltration score, an induced regulatory T cells (iTreg), macrophages, neutrophils, type 1 regulatory T cells (Tr1), type 2 helper T cells (Th2), type 17 helper T cells (Th17), and follicular helper T cells (Tfh).

14. The analytical method of claim 13, wherein the gene expression level is measured by one or more methods selected from the group consisting of reverse transcriptional polymerase chain reaction (RT-PCR), sequencing, RNA sequencing, a microarray, droplet digital polymerase chain reaction (ddPCR), and next generation sequencing (NGS).

15. The analytical method of claim 13, wherein the subject is a cancer patient before or after treatment with cancer immunotherapy.

16. The analytical method of claim 13, wherein the biological sample is one or more selected from the group consisting of tissue, cells, whole blood, serum, plasma, saliva, sputum, cerebrospinal fluid, urine, and stool, isolated from the subject.

17. The analytical method of claim 13, further comprising:
predicting that the subject has good efficacy for cancer immunotherapy or a favorable prognosis, when one or more selected from the group consisting of a TNF score, an immune infiltration score, an abundance score for macrophages, an abundance score for neutrophils, and an abundance score for type 17 helper T cells (Th17) is lower than that of a subject who does not have efficacy for cancer immunotherapy, or
predicting that the subject has good efficacy for cancer immunotherapy or a favorable prognosis, when abundance scores for one or more selected from the group consisting of CD8⁺ T cells (CD8⁺ T), central memory T cells (Tcm), cytotoxic T cells (Tc), induced regulatory T cells (iTreg), type 1 regulatory T cells (Tr1), type 2 helper T cells (Th2), and follicular helper T cells (Tfh) is higher than that of a subject who does not have efficacy for cancer immunotherapy.

18. The analytical method of claim 13, further comprising:
inputting the measured gene expression level of TNF score and/or the immune cell profile to a machine learning-based model; and
predicting efficacy of cancer immunotherapy or a prognosis by automatically classifying the patterns of change compared with the gene expression level of a subject who does not have efficacy for cancer immunotherapy, which is previously input to the machine learning-based model.

19. The analytical method of claim 18, wherein the machine learning-based model is one or more selected from the group consisting of XGBoost and Random forest.

20. A method for treating cancer, comprising:
detecting a tumor necrosis factor (TNF) score and/or an immune cell profile in a biological sample isolated from a subject by measuring a gene expression level, wherein the immune cell profile is one or more selected from the group consisting of CD8⁺ T cells (CD8⁺ T), central memory T cells (Tcm), cytotoxic T cells (Tc), an immune infiltration score, induced regulatory T cells (iTreg), macrophages, neutrophils, type 1 regulatory T cells (Tr1), type 2 helper T cells (Th2), type 17 helper T cells (Th17), and follicular helper T cells (Tfh);
predicting that efficacy or prognosis of cancer immunotherapy is good when one or more selected from the group consisting of a tumor necrosis factor (TNF) score, an immune infiltration score, an abundance score for macrophages, an abundance score for neutrophils, and an abundance score for type 17 helper T cells (Th17) are lower than those of a subject having no efficacy for cancer immunotherapy,
or when abundance scores for one or more selected from the group consisting of CD8⁺ T cells (CD8⁺ T), central memory T cells (Tcm), cytotoxic T cells (Tc), induced regulatory T cells (iTreg), type 1 regulatory T cells (Tr1), type 2 helper T cells (Th2), and follicular helper T cells (Tfh) are higher than those of a subject having no efficacy for cancer immunotherapy; and
treating the subject predicted to have good efficacy or prognosis for cancer immunotherapy with cancer immunotherapy.

21. The method of claim 20, wherein the cancer immunotherapy comprises administering one or more selected from the group consisting of an agent for immune checkpoint blockade (ICB), an immune cell therapeutic agent, a therapeutic antibody, and an immune enhancer.
